(19) United States
(12) Patent Application Publication
(10) Pub. No.: US 2016/0199372 A1
(43) Pub. Date: Jul. 14, 2016

(54) IMIDAZOPYRIDAZINE KINASE INHIBITORS USEFUL TO TREATING A DISEASE OR DISORDER MEDIATED BY AAK1, SUCH AS ALZHEIMER'S DISEASE, BIPOLAR DISORDER, PAIN, SCHIZOPHRENIA

(71) Applicant: Bristol-Myers Squibb Company, Princeton, NJ (US)

(72) Inventors: Richard A. HARTZ, Middletown, CT (US); Vijay T. AHUJA, Princeton, NJ (US); Ramkumar RAJAMANI, Woodbridge, CT (US); Carolyn Diane DZIERBA, Middletown, CT (US); Joanne J. BRONSON, Durham, CT (US); John E. MACOR, Washington Crossing, PA (US)

(21) Appl. No.: 14/912,628

(22) PCT Filed: Aug. 12, 2014
(86) PCT No.: PCT/US2014/050727

§ 371 Date: Feb. 18, 2016

Related U.S. Application Data
(60) Provisional application No. 61/867,638, filed on Aug. 20, 2013.

(51) Int. Cl. A61K 31/5025

(52) U.S. Cl. CPC A61K 31/5025

(57) ABSTRACT

The present disclosure is generally directed to compounds which can inhibit AAK1 (adaptor associated kinase 1), compositions comprising such compounds, and methods for inhibiting AAK1.

This application claims the benefit of U.S. Provisional Application Ser. No. 61/867,638, filed Aug. 20, 2013, which is hereby incorporated by reference in its entirety.

The present disclosure is generally directed to compounds which can inhibit adaptor associated kinase 1 (AAK1), compositions comprising such compounds, and methods for inhibiting AAK1.

Adaptor associated kinase 1 (AAK1) is a member of the Ark1/Prk1 family of serine/threonine kinases. AAK1 mRNA exists in two splice forms termed short and long. The long form predominates and is highly expressed in brain and heart (Henderson and Conner, Mol. Biol. Cell. 2007, 18, 2698-2706). AAK1 is enriched in synaptosomal preparations and is co-localized with endocytic structures in cultured cells. AAK1 modulates clatherin coated endocytosis, a process that is important in synaptic vesicle recycling and receptor-mediated endocytosis. AAK1 associates with the AP2 complex, a hetero-tetramer which links receptor cargo to the clatherin coat. The binding of clatherin to AAK1 stimulates AAK1 kinase activity (Conner et. al., Traffic 2003, 4, 885-890; Jackson et. al., J. Cell. Biol. 2003, 163, 231-236). AAK1 phosphorylates the mu-2 subunit of AP-2, which promotes the binding of mu-2 to tyrosine containing sorting motifs on cargo receptors (Ricotta et. al., J. Cell Bio. 2002, 156, 791-795; Conner and Schmid, J. Cell Bio. 2002, 156, 921-929). Mu2 phosphorylation is not required for receptor uptake, but phosphorylation enhances the efficiency of internalization (Motely et. al., Mol. Biol. Cell. 2006, 17, 5298-5308).

AAK1 has been identified as an inhibitor of Neuregulin-1/ErbB4 signaling in PC12 cells. Loss of AAK1 expression through RNA interference mediated gene silencing or treatment with the kinase inhibitor K252a (which inhibits AAK1 kinase activity) results in the potentiation of Neuregulin-1 induced neurite outgrowth. These treatments result in increased expression of ErbB4 and accumulation of ErbB4 in or near the plasma membrane (Kuai et. al., Chemistry and Biology 2011, 18, 891-906). NRG1 and ErbB4 are putative schizophrenia susceptibility genes (Buonanno, Brain Res. Bull. 2010, 83, 122-131). SNPs in both genes have been associated with multiple schizophrenia endophenotypes (Greenwood et. al., Am. J. Psychiatry 2011, 168, 930-946). Neuregulin 1 and ErbB4 KO mouse models have shown schizophrenia relevant morphological changes and behavioral phenotypes (Jaaro-Peled et. al., Schizophrenia Bulletin 2010, 36, 301-313; Wen et. al., Proc. Natl. Acad. Sci. USA. 2010, 107, 1211-1216). In addition, a single nucleotide polymorphism in an intron of the AAK1 gene has been associated with the age of onset of Parkinson's disease (Latourelle et. al., BMC Med. Genet. 2009, 10, 98). These results suggest that inhibition of AAK1 activity may have utility in the treatment of schizophrenia, cognitive deficits in schizophrenia, Parkinson's disease, neuropathic pain, bipolar disorder, and Alzheimer's disease.

In a first aspect the present disclosure provides a method for treating or managing a disease or a disorder mediated by AAK1 activity, the method comprising administering to a patient in need thereof a therapeutically effective amount of a compound of formula (I)

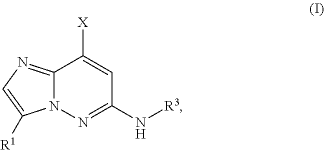

or a pharmaceutically acceptable salt thereof, wherein:

R¹ is selected from —C(O)NHR² and thienyl;

R² is selected from

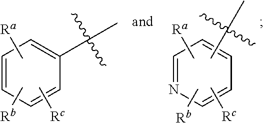

wherein R^{a} and R^{b} are independently selected from hydrogen, C₂-C₄ alkenyl, C₁-C₃alkoxy, C₁-C₃alkoxyC₁-C₃alkyl, C₁-C₃alkyl, cyano, halo, C₁-C₃ haloalkyl, hydroxy, and C₁-C₃hydroxyalkyl; or, alternatively,

when R^{a} and R^{b} are on adjacent carbons, they, together with the carbon atoms to which they are attached, can optionally form a five-membered aromatic ring containing one or two nitrogen atoms;

R^{c} is a five-membered aromatic ring containing one, two, three, or four heteroatoms independently selected from nitrogen, oxygen, and sulfur, wherein the five-membered aromatic ring is optionally substituted with one group selected from C₁-C₄alkoxy, C₁-C₄ alkoxyC₁-C₄alkyl, C₁-C₄alkyl, C₁-C₄ aminoalkyl, cyano, C₃-C₆ cycloalkyl, C₁-C₄ haloalkyl, C₁-C₄ hydroxyalkyl, nitro, and phenyl;

R³ is selected from 4-(C₁-C₃acylamino)cyclohexyl, C₁-C₄-aminoalkyl, 2-aminocyclobutyl, 4-aminocyclohexyl, 3-aminocyclopentyl, 3-aminomethylcyclohexyl, 3-aminomethylcyclopentyl, 2-cyanocyclobutyl, 4-cyanocyclohexyl, cyanomethyl, 2-methylaminocyclobutyl, 4-methylaminocyclohexyl, 3-methylaminocyclopentyl, octahydrocyclopenta[c]pyrrolyl, 4-piperidyl, and 3-azabicyclo[3.2.1]octyl; and

X is selected from hydrogen, C₁-C₃alkylamino, C₃-C₆cycloalkylamino, and phenylamino, wherein the phenylamino is optionally substituted with one group selected from C₁-C₃alkoxy, C₁-C₃alkyl, cyano, and a five-membered aromatic ring containing one, two, or three heteroatoms independently selected from nitrogen, oxygen, and sulfur wherein the five-membered aromatic ring is optionally substituted with one C₁-C₃alkyl group.

In a first embodiment of the first aspect the present disclosure provides a method for treating or managing a disease or a disorder mediated by AAK1 activity, the method comprising administering to a patient in need thereof a therapeutically effective amount of a compound of formula (I), or a pharmaceutically acceptable salt thereof, wherein

R² is

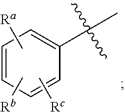

wherein R^{a} and R^{b} are hydrogen;

R^{c} is a five-membered aromatic ring containing one, two, three, or four heteroatoms independently selected from nitrogen, oxygen, and sulfur, wherein the five-membered aromatic ring is optionally substituted with one group selected from C₁-C₄alkoxy, cyano, nitro, and phenyl; and

R³ is 4-aminocyclohexyl.

In a second embodiment of the first aspect the present disclosure provides a method for treating or managing a disease or a disorder mediated by AAK1 activity, the method comprising administering to a patient in need thereof a therapeutically effective amount of a compound of formula (I), or a pharmaceutically acceptable salt thereof, wherein the disease or disorder is selected from Alzheimer's disease, bipolar disorder, pain, Parkinson's disease, and schizophrenia. In a third embodiment the pain is neuropathic pain. In a fourth embodiment the neuropathic pain is fibromyalgia or peripheral neuropathy.

In a second aspect the present disclosure provides a method of inhibiting adaptor associated kinase 1 (AAK1) activity, comprising contacting AAK1 with a compound of formula (I)

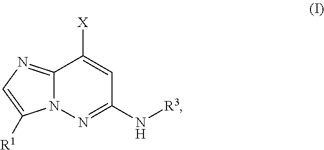

or a pharmaceutically acceptable salt thereof, wherein:

R¹ is selected from —C(O)NHR² and thienyl;

R² is selected from

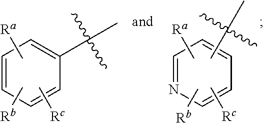

wherein R^{a} and R^{b} are independently selected from hydrogen, C₂-C₄ alkenyl, C₁-C₃alkoxy, C₁-C₃alkoxyC₁-C₃alkyl, C₁-C₃ alkyl, cyano, halo, C₁-C₃ haloalkyl, hydroxy, and C₁-C₃hydroxyalkyl; or, alternatively,

when R^{a} and R^{b} are on adjacent carbons, they, together with the carbon atoms to which they are attached, can optionally form a five-membered aromatic ring containing one or two nitrogen atoms;

R^{c} is a five-membered aromatic ring containing one, two, three, or four heteroatoms independently selected from nitrogen, oxygen, and sulfur, wherein the five-membered aromatic ring is optionally substituted with one group selected from C₁-C₄alkoxy, C₁-C₄ alkoxyC₁-C₄alkyl, C₁-C₄alkyl, C₁-C₄ aminoalkyl, cyano, C₃-C₆ cycloalkyl, C₁-C₄ haloalkyl, C₁-C₄ hydroxyalkyl, nitro, and phenyl;

R³ is selected from 4-acylaminocyclohexyl, C₁-C₄-aminoalkyl, 2-aminocyclobutyl, 4-aminocyclohexyl, 3-aminocyclopentyl, 3-aminomethylcyclohexyl, 3-aminomethylcyclopentyl, 2-cyanocyclobutyl, 4-cyanocyclohexyl, cyanomethyl, 2-methylaminocyclobutyl, 4-methylaminocyclohexyl, 3-methylaminocyclopentyl, octahydrocyclopenta[c]pyrrolyl, 4-piperidyl, and 3-azabicyclo[3.2.1]octyl; and

X is selected from hydrogen, C₁-C₃alkylamino, C₃-C₆cycloalkylamino, and phenylamino, wherein the phenylamino is optionally substituted with one group selected from C₁-C₃alkoxy, C₁-C₃alkyl, cyano, and a five-membered aromatic ring containing one, two, or three heteroatoms independently selected from nitrogen, oxygen, and sulfur wherein the five-membered aromatic ring is optionally substituted with one C₁-C₃alkyl group.

In a first embodiment of the second aspect the present disclosure provides a method for treating or managing a disease or a disorder mediated by AAK1 activity, the method comprising administering to a patient in need thereof a therapeutically effective amount of a compound of formula (I), or a pharmaceutically acceptable salt thereof, wherein

R² is

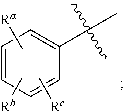

wherein R^{a} and R^{b} are hydrogen;

R^{c} is a five-membered aromatic ring containing one, two, three, or four heteroatoms independently selected from nitrogen, oxygen, and sulfur, wherein the five-membered aromatic ring is optionally substituted with one group selected from C₁-C₄alkoxy, cyano, nitro, and phenyl; and

R³ is 4-aminocyclohexyl.

Other aspects of the present disclosure may include suitable combinations of embodiments disclosed herein.

Yet other aspects and embodiments may be found in the description provided herein.

### BRIEF DESCRIPTION OF THE FIGURES

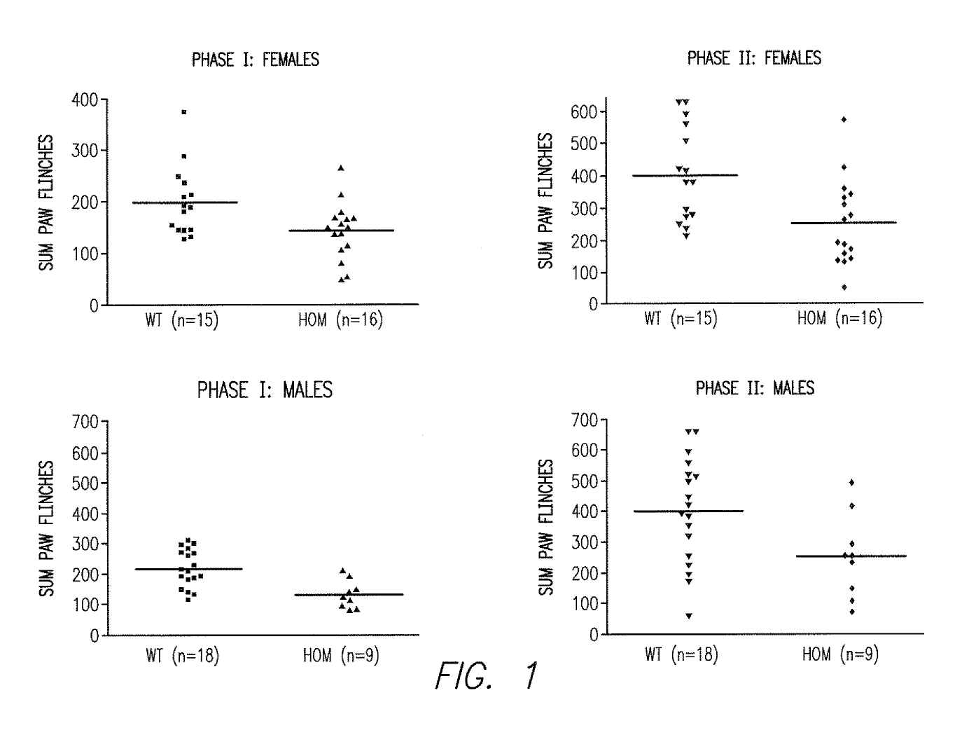

Aspects of the disclosure are illustrated in FIG. 1, which shows results obtained from a formalin pain model using AAK1 homozygous (−/−) knockout mice and their wild-type (+/+) littermates. The AAK1 homozygous (−/−) knockout mice show a clear reduction in both acute and tonic pain response as compared to their wild-type (+/+) littermates.

This disclosure is based, in part, on the discovery that AAK1 knockout mice exhibit a high resistance to pain. That discovery prompted research that ultimately led to the discovery of AAK1 inhibitors, compositions comprising them, and methods of their use.

The description of the present disclosure herein should be construed in congruity with the laws and principals of chemical bonding. In some instances it may be necessary to remove a hydrogen atom in order to accommodate a substituent at any given location.

It should be understood that the compounds encompassed by the present disclosure are those that are suitably stable for use as pharmaceutical agent.

As used in the present specification, the following terms have the meanings indicated:

All patents, patent applications, and literature references cited in the specification are herein incorporated by reference in their entirety. In the case of inconsistencies, the present disclosure, including definitions, will prevail.

As used herein, the singular forms “a”, “an”, and “the” include plural reference unless the context clearly dictates otherwise.

In some instances, the number of carbon atoms in any particular group is denoted before the recitation of the group. For example, the term “C₁-6 alkyl” denotes an alkyl group containing one to six carbon atoms. Where these designations exist they supercede all other definitions contained herein.

The term “acyl,” as used herein, refers to —C(O)R, wherein R is an alkyl group.

The term “acylamino,” as used herein, refers to —NHR wherein R is an acyl group.

The term “alkenyl,” as used herein, refers to The term “alkenyl,” as used herein, refers to a straight or branched chain group containing at least one carbon-carbon double bond.

The term “alkoxy,” as used herein, refers to an alkyl group attached to the parent molecular moiety through an oxygen atom.

The term “alkoxyalkyl,” as used herein, refers to an alkyl group substituted with one, two, or three alkoxy groups.

The term “alkyl,” as used herein, refers to a group derived from a straight or branched chain saturated hydrocarbon.

The term “alkylamino,” as used herein refers to —NHR, wherein R is an alkyl group.

The term “amino,” as used herein, refers to —NH₂.

The term “aminoalkyl,” as used herein, refers to an alkyl group substituted by one, two, or three amino groups.

The term “cyano,” as used herein, refers to —CN.

The term “cycloalkyl,” as used herein, refers to a saturated monocyclic hydrocarbon ring system having zero heteroatoms. Representative examples of cycloalkyl groups include, but are not limited to, cyclopropyl, cyclopentyl, and cyclohexyl.

The term “cycloalkylamino,” as used herein, refers to —NHR wherein R is a cycloalkyl group.

The term “halo,” as used herein, refers to Br, Cl, F, and/or I.

The term “haloalkyl,” as used herein, refers to an alkyl group substituted by one, two, three, or four halogen atoms.

The term “hydroxy,” as used herein, refers to —OH.

The term “hydroxyalkyl,” as used herein, refers to a hydroxy group attached to the parent molecular moiety through an alkyl group.

The term “nitro,” as used herein, refers to —NO₂.

Asymmetric centers may exist in the compounds of the present disclosure. It should be understood that the disclosure encompasses all stereochemical isomeric forms, or mixtures thereof, which possess the ability to inhibit AAK1. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure may also exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

The term “compounds of the present disclosure”, and equivalent expressions, are meant to embrace compounds of formula (I), and pharmaceutically acceptable enantiomers, diastereomers, and salts thereof. Similarly, references to intermediates are meant to embrace their salts where the context so permits.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

The compounds of the present disclosure can exist as pharmaceutically acceptable salts. The term “pharmaceutically acceptable salt,” as used herein, represents salts or zwitterionic forms of the compounds of the present disclosure which are water or oil-soluble or dispersible, which are, within the scope of sound medical judgment, suitable for use in contact with the tissues of patients without excessive toxicity, irritation, allergic response, or other problem or complication commensurate with a reasonable benefit/risk ratio, and are effective for their intended use. The salts can be prepared during the final isolation and purification of the compounds or separately by reacting a suitable nitrogen atom with a suitable acid. Representative acid addition salts include acetate, adipate, alginate, citrate, aspartate, benzoate, benzenesulfonate, bisulfate, butyrate, camphorate, camphorsulfonate; digluconate, dihydrobromide, diydrochloride, dihydroiodide, glycerophosphate, hemisulfate, heptanoate, hexanoate, formate, fumarate, hydrochloride, hydrobromide, hydroiodide, 2-hydroxyethanesulfonate, lactate, maleate, mesitylenesulfonate, methanesulfonate, naphthylenesulfonate, nicotinate, 2-naphthalenesulfonate, oxalate, palmoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Examples of acids which can be employed to form pharmaceutically acceptable addition salts include inorganic acids such as hydrochloric, hydrobromic, sulfuric, and phosphoric, and organic acids such as oxalic, maleic, succinic, and citric.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, and N,N′-dibenzylethylenediamine. Other representative organic amines useful for the formation of base addition salts include ethylenediamine, ethanolamine, diethanolamine, piperidine, and piperazine.

One embodiment of this disclosure encompasses methods of inhibiting adaptor associated kinase 1 (AAK1), both in vitro and in vivo, which comprise contacting AAK1 with a compound of formula I or a pharmaceutically acceptable salt thereof.

When it is possible that, for use in therapy, therapeutically effective amounts of a compound of formula (I), as well as pharmaceutically acceptable salts thereof, may be administered as the raw chemical, it is possible to present the active ingredient as a pharmaceutical composition. Accordingly, the disclosure further provides pharmaceutical compositions, which include therapeutically effective amounts of compounds of formula (I) or pharmaceutically acceptable salts thereof, and one or more pharmaceutically acceptable carriers, diluents, or excipients. Unless otherwise indicated, a “therapeutically effective amount” of a compound is an amount sufficient to provide a therapeutic benefit in the treatment or management of a disease or condition, or to delay or minimize one or more symptoms associated with the disease or condition. The term “therapeutically effective amount” can encompass an amount that improves overall therapy, reduces or avoids symptoms or causes of a disease or condition, or enhances the therapeutic efficacy of another therapeutic agent.

The term “therapeutically effective amount,” as used herein, refers to an amount of a compound or compounds sufficient to provide a therapeutic benefit in the treatment or management of a disease or condition, or to delay or minimize one or more symptoms associated with the disease or condition. A “therapeutically effective amount” of a compound means an amount of therapeutic agent, alone or in combination with other therapies, that provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent. When applied to an individual active ingredient, administered alone, the term refers to that ingredient alone. When applied to a combination, the term refers to combined amounts of the active ingredients that result in the therapeutic effect, whether administered in combination, serially, or simultaneously. The compounds of formula (I) and pharmaceutically acceptable salts thereof, are as described above. The carrier(s), diluent(s), or excipient(s) must be acceptable in the sense of being compatible with the other ingredients of the formulation and not deleterious to the recipient thereof. In accordance with another aspect of the present disclosure there is also provided a process for the preparation of a pharmaceutical formulation including admixing a compound of formula (I), or a pharmaceutically acceptable salt thereof, with one or more pharmaceutically acceptable carriers, diluents, or excipients. The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

Pharmaceutical formulations may be presented in unit dose forms containing a predetermined amount of active ingredient per unit dose. Dosage levels of between about 0.01 and about 250 milligram per kilogram (“mg/kg”) body weight per day, preferably between about 0.05 and about 100 mg/kg body weight per day of the compounds of the present disclosure are typical in a monotherapy for the prevention and treatment of disease. Typically, the pharmaceutical compositions of this disclosure will be administered from about 1 to about 5 times per day or alternatively, as a continuous infusion. Such administration can be used as a chronic or acute therapy. The amount of active ingredient that may be combined with the carrier materials to produce a single dosage form will vary depending on the condition being treated, the severity of the condition, the time of administration, the route of administration, the rate of excretion of the compound employed, the duration of treatment, and the age, gender, weight, and condition of the patient. Preferred unit dosage formulations are those containing a daily dose or sub-dose, as herein above recited, or an appropriate fraction thereof, of an active ingredient. Treatment may be initiated with small dosages substantially less than the optimum dose of the compound. Thereafter, the dosage is increased by small increments until the optimum effect under the circumstances is reached. In general, the compound is most desirably administered at a concentration level that will generally afford effective results without causing any harmful or deleterious side effects.

When the compositions of this disclosure comprise a combination of a compound of the present disclosure and one or more additional therapeutic or prophylactic agent, both the compound and the additional agent are usually present at dosage levels of between about 10 to 150%, and more preferably between about 10 and 80% of the dosage normally administered in a monotherapy regimen.

Compounds of the disclosure may be administered in combination with one or more additional therapeutic or prophylactic agents. For example, when used for the treatment of pain, possible additional agents include immunosuppressive agents, anti-inflammatory agents, and/or other agents used in the treatment of pain.

Immunosuppressants suitable for use in the methods and compositions of this disclosure include those known in the art. Examples include aminopterin, azathioprine, cyclosporin A, D-penicillamine, gold salts, hydroxychloroquine, leflunomide, methotrexate, minocycline, rapamycin, sulfasalazine, tacrolimus (FK506), and pharmaceutically acceptable salts thereof. A particular immunosuppressant is methotrexate.

Additional examples of immunosuppressants include anti-TNF antibodies, such as adalimumab, certolizumab pegol, etanercept, and infliximab. Others include interleukin-1 blockers, such as anakinra Others include anti-B cell (CD20) antibodies, such as rituximab. Others include T cell activation blockers, such as abatacept.

Other immunosuppressants include inosine monophosphate dehydrogenase inhibitors, such as mycophenolate mofetil (CellCept®) and mycophenolic acid (Myfortic®).

Anti-inflammatory drugs suitable for use in the methods and compositions of this disclosure include those known in the art. Examples include glucocorticoids and NSAIDs. Examples of glucocorticoids include aldosterone, beclometasone, betamethasone, cortisone, deoxycorticosterone, dexamethasone, fludrocortisones, hydrocortisone, methylprednisolone, prednisolone, prednisone, triamcinolone, and pharmaceutically acceptable salts thereof.

Examples of NSAID include salicylates (e.g., aspirin, amoxiprin, benorilate, choline magnesium salicylate, diflunisal, faislamine, methyl salicylate, magnesium salicylate, salicyl salicylate, and pharmaceutically acceptable salts thereof), arylalkanoic acids (e.g., diclofenac, aceclofenac, acemetacin, bromfenac, etodolac, indometacin, nabumetone, sulindac, tolmetin, and pharmaceutically acceptable salts thereof), arylpropionic acids (e.g., ibuprofen, carprofen, fenbufen, fenoprofen, flurbiprofen, ketoprofen, ketorolac, loxoprofen, naproxen, oxaprozin, tiaprofenic acid, suprofen, and pharmaceutically acceptable salts thereof), arylanthranilic acids (e.g., meclofenamic acid, mefenamic acid, and pharmaceutically acceptable salts thereof), pyrazolidine derivatives (e.g., azapropazone, metamizole, oxyphenbutazone, phenylbutazone, sulfinprazone, and pharmaceutically acceptable salts thereof), oxicams (e.g., lornoxicam, meloxicam, piroxicam, tenoxicam, and pharmaceutically acceptable salts thereof), COX-2 inhibitors (e.g., celecoxib, etoricoxib, lumiracoxib, parecoxib, rofecoxib, valdecoxib, and pharmaceutically acceptable salts thereof), and sulphonanilides (e.g., nimesulide and pharmaceutically acceptable salts thereof).

Other agents used in the treatment of pain (including but not limited to neuropathic and inflammatory pain) include, but are not limited to, agents such as pregabalin, lidocaine, duloxetine, gabapentin, carbamazepine, capsaicin, and other serotonin/norepinephrine/dopamine reuptake inhibitors, and opiates (such as oxycontin, morphine, and codeine).

In the treatment of pain caused by a known disease or condition, such as diabetes, infection (e.g., herpes zoster or HIV infection), or cancer, compounds of the disclosure may be administered in combination with one or more additional therapeutic or prophylactic agents directed at the underlying disease or condition. For example, when used to treat diabetic neuropathy, compounds of the disclosure may be administered in combination with one or more anti-diabetic agents, anti-hyperglycemic agents, hypolipidemic/lipid lowering agents, anti-obesity agents, anti-hypertensive agents and appetite suppressants. Examples of anti-diabetic agents include biguanides (e.g., metformin, phenformin), glucosidase inhibitors (e.g., acarbose, miglitol), insulins (including insulin secretagogues and insulin sensitizers), meglitinides (e.g., repaglinide), sulfonylureas (e.g., glimepiride, glyburide, gliclazide, chlorpropamide, and glipizide), biguanide/glyburide combinations (e.g., Glucovance), thiazolidinediones (e.g., troglitazone, rosiglitazone, and pioglitazone), PPAR-alpha agonists, PPAR-gamma agonists, PPAR alpha/gamma dual agonists, glycogen phosphorylase inhibitors, inhibitors of fatty acid binding protein (aP2), glucagon-like peptide-1 (GLP-1) or other agonists of the GLP-1 receptor, dipeptidyl peptidase IV (DPP4) inhibitors, and sodium-glucose co-transporter 2 (SGLT2) inhibitors (e.g., dapagliflozin, canagliflozin, and LX-4211).

Pharmaceutical formulations may be adapted for administration by any appropriate route, for example by the oral (including buccal or sublingual), rectal, nasal, topical (including buccal, sublingual, or transdermal), vaginal, or parenteral (including subcutaneous, intracutaneous, intramuscular, intra-articular, intrasynovial, intrasternal, intrathecal, intralesional, intravenous, or intradermal injections or infusions) route. Such formulations may be prepared by any method known in the art of pharmacy, for example by bringing into association the active ingredient with the carrier(s) or excipient(s). Oral administration or administration by injection are preferred.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil emulsions.

For instance, for oral administration in the form of a tablet or capsule, the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing, and coloring agent can also be present.

Capsules are made by preparing a powder mixture, as described above, and filling formed gelatin sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate, or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, and the like. Lubricants used in these dosage forms include sodium oleate, sodium chloride, and the like. Disintegrators include, without limitation, starch, methyl cellulose, agar, betonite, xanthan gum, and the like. Tablets are formulated, for example, by preparing a powder mixture, granulating or slugging, adding a lubricant and disintegrant, and pressing into tablets. A powder mixture is prepared by mixing the compound, suitable comminuted, with a diluent or base as described above, and optionally, with a binder such as carboxymethylcellulose, an aliginate, gelating, or polyvinyl pyrrolidone, a solution retardant such as paraffin, a resorption accelerator such as a quaternary salt and/or and absorption agent such as betonite, kaolin, or dicalcium phosphate. The powder mixture can be granulated by wetting with a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds of the present disclosure can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solution, syrups, and elixirs can be prepared in dosage unit form so that a given quantity contains a predetermined amount of the compound. Syrups can be prepared by dissolving the compound in a suitably flavored aqueous solution, while elixirs are prepared through the use of a non-toxic vehicle. Solubilizers and emulsifiers such as ethoxylated isostearyl alcohols and polyoxyethylene sorbitol ethers, preservatives, flavor additive such as peppermint oil or natural sweeteners, or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax, or the like.

The compounds of formula (I), and pharmaceutically acceptable salts thereof, can also be administered in the form of liposome delivery systems, such as small unilamellar vesicles, large unilamellar vesicles, and multilamellar vesicles. Liposomes can be formed from a variety of phopholipids, such as cholesterol, stearylamine, or phophatidylcholines.

The compounds of formula (I) and pharmaceutically acceptable salts thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research 1986, 3(6), 318.

Pharmaceutical formulations adapted for topical administration may be formulated as ointments, creams, suspensions, lotions, powders, solutions, pastes, gels, sprays, aerosols, or oils.

Pharmaceutical formulations adapted for rectal administration may be presented as suppositories or as enemas.

Pharmaceutical formulations adapted for nasal administration wherein the carrier is a solid include a course powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers, or insufflators.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and soutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It should be understood that in addition to the ingredients particularly mentioned above, the formulations may include other agents conventional in the art having regard to the type of formulation in question, for example those suitable for oral administration may include flavoring agents.

The term “patient” includes both human and other mammals.

Unless otherwise indicated, the terms “manage,” “managing”, and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

The term “treating” refers to: (i) preventing a disease, disorder or condition from occurring in a patient that may be predisposed to the disease, disorder, and/or condition but has not yet been diagnosed as having it; (ii) inhibiting the disease, disorder, or condition, i.e., arresting its development; and (iii) relieving the disease, disorder, or condition, i.e., causing regression of the disease, disorder, and/or condition.

This disclosure is intended to encompass compounds having Formula (I) when prepared by synthetic processes or by metabolic processes including those occurring in the human or animal body (in vivo) or processes occurring in vitro. The abbreviations used in the present application, including particularly in the illustrative schemes and examples which follow, are well-known to those skilled in the art. Some of the abbreviations used are as follows: CDI (N,N′-carbonyldiimidazole); DIEA or i-Pr₂NEt for diisopropylethylamine; DMF for N,N-dimethylformamide; THF for tetrahydrofuran; HATU for O-(7-azabenzotriazol-1-yl)-N,N,N′,N′-tetramethyluronium hexafluorophosphate; BOP for benzotriazol-1-yloxytris(dimethylamino)phosphonium hexafluorophosphate; EDC or EDCI for 1-(3-dimethylaminopropyl)-3-ethylcarbodiimide hydrochloride; TBTU for O-benzotriazol-1-yl-N,N,N′,N′-tetramethyluronium tetrafluoroborate; LiHMDS for lithium hexamethyldisilazide; PMB forpara-methoxybenzyl; n-BuLi for n-butyllithium; TFA for trifluoroacetic acid; EtOH for ethanol; NBS for N-bromosuccinimide; NIS for N-iodosuccinimide; NCS for N-chlorosuccinimide; dba for dibenzylideneacetone; Et for ethyl; Ph for phenyl; MeOH for methanol; Me for methyl; min or mins for minutes; h or hr for hours; RT or rt or r.t. for room temperature or retention time (context will dictate); and t_{R} for retention time.

### EXAMPLES

The present disclosure will now be described in connection with certain embodiments which are not intended to limit its scope. On the contrary, the present disclosure covers all alternatives, modifications, and equivalents as can be included within the scope of the claims. Thus, the following examples, which include specific embodiments, will illustrate one practice of the present disclosure, it being understood that the examples are for the purposes of illustration of certain embodiments and are presented to provide what is believed to be the most useful and readily understood description of its procedures and conceptual aspects.

The compounds of the present disclosure may be prepared using the reactions and techniques described in this section as well as other synthetic methods known to those of ordinary skill in the art. The reactions are performed in solvents appropriate to the reagents and materials employed and suitable for the transformation being effected. Also, in the description of the synthetic methods described below, it is to be understood that all proposed reaction conditions, including choice of solvents, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents which are compatible with the reaction conditions will be readily apparent to one skilled in the art and alternate methods must then be used.

Representative schemes for the preparation of intermediates used in the synthesis of compounds of formula (I) are shown below. Intermediate amines 5, 10, 13, 17, and 19 can be prepared by the routes shown in Schemes 1-5.

Intermediates of formula 5 are prepared by the methods outlined in Scheme 1. Treatment of 2 with hydroxylamine hydrochloride in pyridine affords compound 3. Compound 3 can be coupled with an acid chloride, either in the presence or absence of a reagent to promote the coupling reaction, in a solvent such as pyridine. If a coupling agent is used, a reagent such as 1,1′-carbonyldiimidazole can be used. Subsequent heating of the reaction mixture at temperatures ranging from 80° C. to 140° C. affords compounds of formula 4. Reduction of the nitro group in 4 is accomplished using standard conditions such as, but not limited to, H₂ and Pd/C, zinc with ammonium chloride, or tin chloride, preferably zinc with ammonium chloride, in an appropriate solvent such as methanol or ethanol at temperatures ranging from 0° C. to 100° C. to give compounds of formula 5.

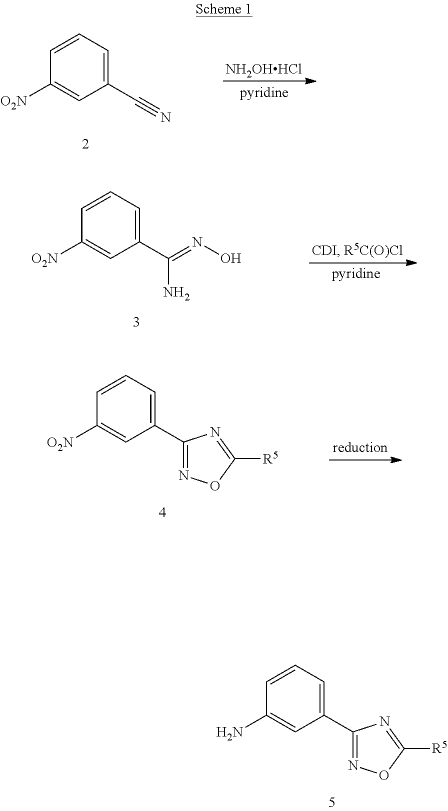

Intermediates of formula 10 are prepared by the methods outlined in Scheme 2. Coupling of acid chloride 6 with acid hydrizides 7 in the presence of a base such as N, N-diisopropylethylamine or triethylamine affords compounds of formula 8. Treatment of compounds of formula 8 with Lawesson's reagent followed by heating at 100° C. furnishes compounds of formula 9. Reduction of the nitro group in 4 as described in Scheme 1 provides compounds of formula 10.

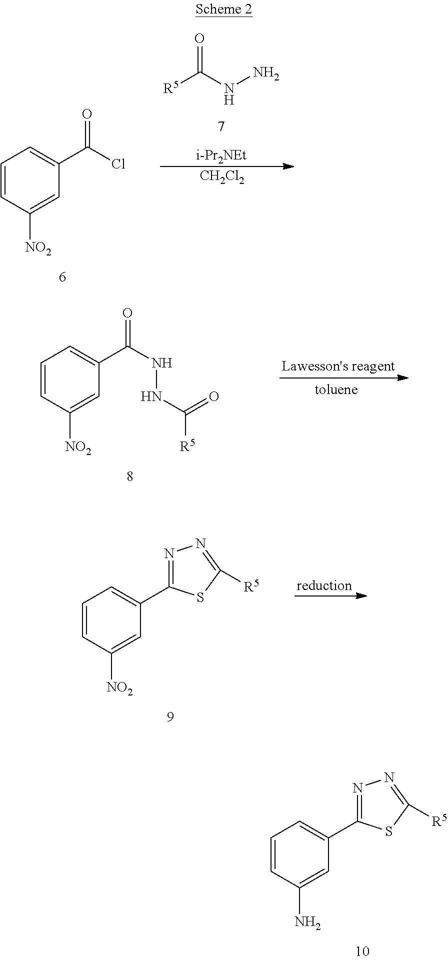

Intermediates of formula 13 are prepared by the methods outlined in Scheme 3. Heating bromomethylketone 11 in the presence of an amide at temperatures ranging from 100° C. to 180° C. in the presence or absence of a solvent, preferably neat, affords compounds of formula 12. Reduction of the nitro group in 12 as described in Scheme 1 provides compounds of formula 13.

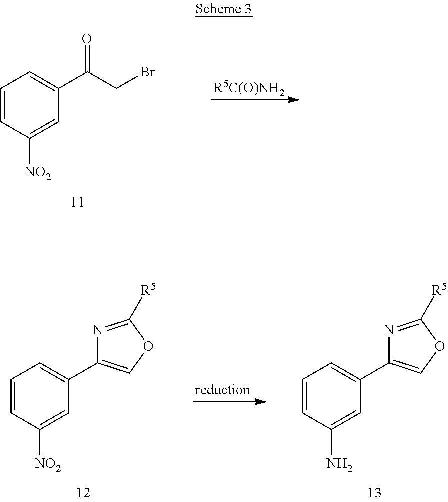

Intermediates of formula 17 are prepared by the methods outlined in Scheme 4. Treatment of compound 14 with sodium azide in a solvent such as DMF at temperatures ranging from 100° C. to 150° C. affords compound 15. Treatment of compounds of formula 15 with a base such as potassium carbonate in the presence of an alkylating agent such as an alkyl halide (LG=halo or other suitable leaving group) in a solvent such as DMF, THF, acetone, or acetonitrile at temperatures ranging from 80° C. to 150° C. furnishes compounds of formula 16. Reduction of the nitro group in 16 as described in Scheme 1 provides compounds of formula 17.

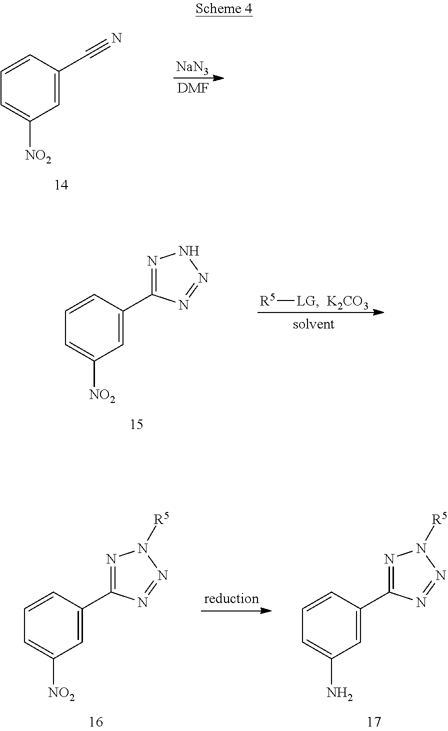

Intermediates of formula 19 are prepared by the methods outlined in Scheme 5. Heating bromomethylketone 11 in the presence of an alkylthioamide at temperatures ranging from 50° C. to 120° C. in a solvent such as ethanol affords compounds of formula 18. Reduction of the nitro group in 18 as described in Scheme 1 provides compounds of formula 19.

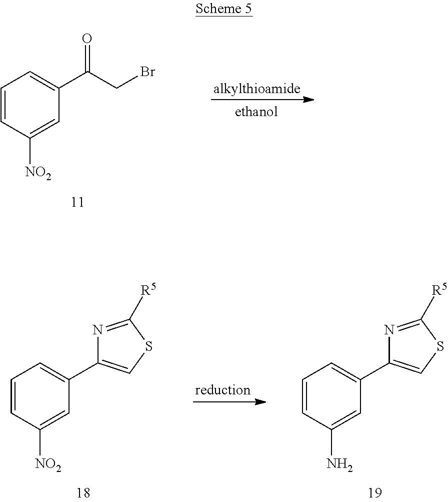

Compounds of formula (I) wherein R¹═—C(O)NHR² and X is a substituted amine are prepared by the method outlined in Scheme 6. Reaction of 20 (Vaccaro, W. et al. United States Patent Appl. US 2008/0045536 A1, 2008) with a suitable amine wherein the amine is substituted with an appropriate protecting group as described in Protective Groups in Organic Synthesis (Greene, Wuts; 3^{rd} ed., 1999, John Wiley & Sons, Inc.), preferably p-methoxybenzyl, in the presence of a base and a solvent such as THF affords 6-haloimidazo[1,2-b]pyridazines 21. The p-methoxybenzyl amine used for reaction with 6,8-dihaloimidazo[1,2-b]pyridines 20 can be prepared using conditions described by Brussee et al. (Brussee, J.; van Benthem, R. A. T. M.; Kruse, C. G.; van der Gen, A. Tetrahedron: Asymmetry 1990, 1, 163) wherein p-methoxybenzaldehyde and an aniline are combined in the presence of a Lewis acid such as magnesium perchlorate and a reducing agent such as sodium borohydride in a solvent system consisting of dichloromethane and methanol or similar solvents. Reaction of 21 with a strong base such as n-butyllithium or t-butyllithium followed by the addition of CO₂, preferably in the solid form (dry ice) in a solvent such as THF at temperatures ranging from −78° C. to room temperature provides 6-haloimidazo[1,2-b]pyridazines 22. Coupling of 6-haloimidazo[1,2-b]pyridazines 22 with an amine, such as compounds 5, 10, 13, 17, or 19, using standard peptide coupling reagents such as HATU, BOP, EDC, or TBTU, preferably HATU or TBTU, in the presence of a base such as N,N-diisopropylethylamine, and a solvent such as dichloromethane, dichloroethane, tetrahydrofuran, or dimethylformamide at temperatures ranging from 0° C. to the boiling point of the solvent (but generally below 80° C.) furnished 6-haloimidazo[1,2-b]pyridazines 23. Reaction of 6-haloimidazo[1,2-b]pyridazines 23 with nuclephiles, such as amines, in either a solvent such as N-methylpyrrolidinone in the presence or absence of a suitable base such as cesium carbonate, or neat (preferably neat) at temperatures ranging from 50° C. to 280° C. using either conventional heating methods or a microwave heating in a manner similar to that previously described (Vaccaro, W. et al. United States Patent Appl. US 2008/0045536 A1, 2008) provides imidazo[1,2-b]pyridazines 24. Removal of the protecting group, preferably p-methoxybenzyl, using conditions described in Protective Groups in Organic Synthesis (Greene, Wuts; 3^{rd} ed., 1999, John Wiley & Sons, Inc.), preferably trifluoroacetic acid, provides imidazo[1,2-b]pyridazines of formula (Ia).

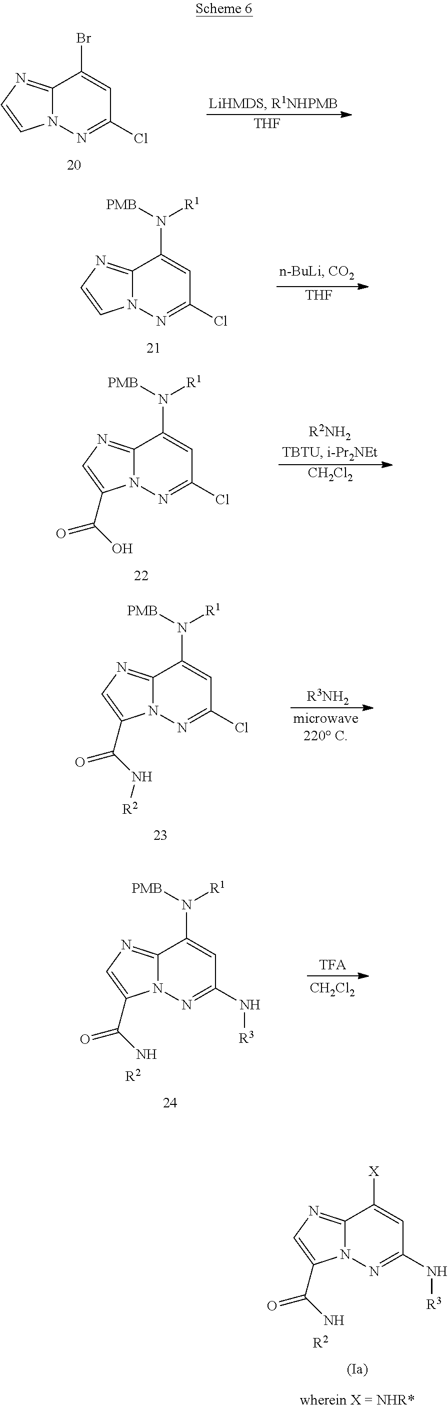

Compounds of formula (I) wherein X═H, R¹ is —C(O)NHR² are prepared by the method outlined in Scheme 7. Condensation of 25 with ethyl 2-chloro-3-oxopropanoate (26) (Larsson, L.; Tammelin, L. E. Acta Chem. Scand. 1961, 15, 349., Yoffe, S. T.; Petrovshky, P. V.; Goryonov, Y. E.; Yershova, T. V.; Kabachni, M. I. Tetrahedron, 1972, 28, 2783) in a solvent such as methanol or ethanol affords 6-haloimidazo[1,2-b]pyridines 27. Hydrolysis of the ester in 27 under either acidic or basic conditions provides 6-chloroimidazo[1,2-b]pyridazine-3-carboxylic acids 28. Coupling of 6-chloroimidazo[1,2-b]pyridazine-3-carboxylic acids 28 with an amine, such as compounds 5, 10, 13, 17, or 19, using standard peptide coupling reagents such as HATU, BOP, EDC, or TBTU, preferably HATU or TBTU, in the presence of a base such as N,N-diisopropylethylamine and a solvent such as dichloromethane, dichloroethane, tetrahydrofuran, or dimethylformamide at temperatures ranging from 0° C. to the boiling point of the solvent (but generally below 80° C. furnished 6-haloimidazo[1,2-b]pyridazines 29. Reaction of 6-haloimidazo[1,2-b]pyridazines 29 with nuclephiles, such as amines, in either a solvent such as N-methylpyrrolidinone in the presence or absence of a suitable base such as cesium carbonate, or neat (preferably neat) at temperatures ranging from 50° C. to 280° C. using either conventional heating methods or a microwave heating provides imidazo[1,2-b]pyridazines (Ia) wherein X═H.

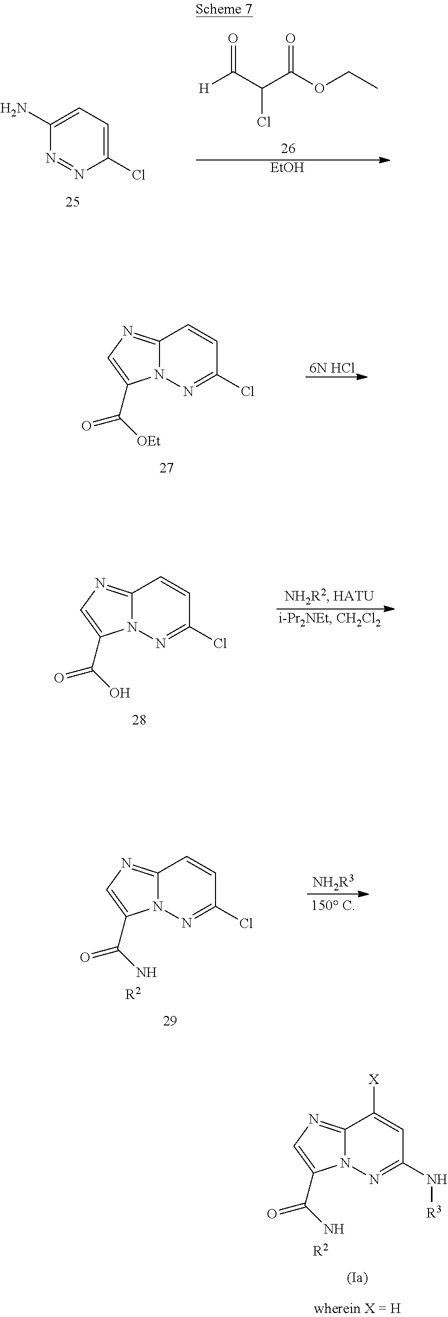

Compounds of formula (I) wherein X is a substituted amine and R¹ is thienyl are prepared by the method outlined in Scheme 8. Halogenation of 20 with halogenating agents such as NIS, NBS, or NCS, preferably NBS, in a solvent such as dichloromethane as described by Vaccaro W. et al. (Vaccaro W. et al. United States Patent Appl. US 2008/0045536 A1, 2008) provides 3,8-dibromo-6-chloroimidazo[1,2-b]pyridazines 30. Treatment of 30 with sodium methoxide in ethanol affords 31. Coupling of 31 with an aryl or heteroaryl boronic acid such as 3-thiophene boronic acid in the presence of a palladium catalyst such as, but not limited to, Pd(PPh₃)₄, Pd₂(dba)₃, or PdCl₂(PPh₃)₂, and a base such as sodium carbonate, potassium carbonate, sodium t-butoxide, potassium acetate, or potassium fluoride in a solvent such as toluene, DMF, THF, ethanol, methanol, water, or a combination of such solvents furnishes 3-substituted-6-chloroimidazo[1,2-b]pyridazines 32. The reaction of 32 with a suitable amine in the presence of a base such as LiHMDS and a solvent such as THF affords 6-haloimidazo[1,2-b]pyridazines 33. Reaction of 6-haloimidazo[1,2-b]pyridazines 33 with nuclephiles, such as amines, in either a solvent such as N-methylpyrrolidinone in the presence or absence of a suitable base such as cesium carbonate, or neat (preferably neat) provides the imidazo[1,2-b]pyridazines.

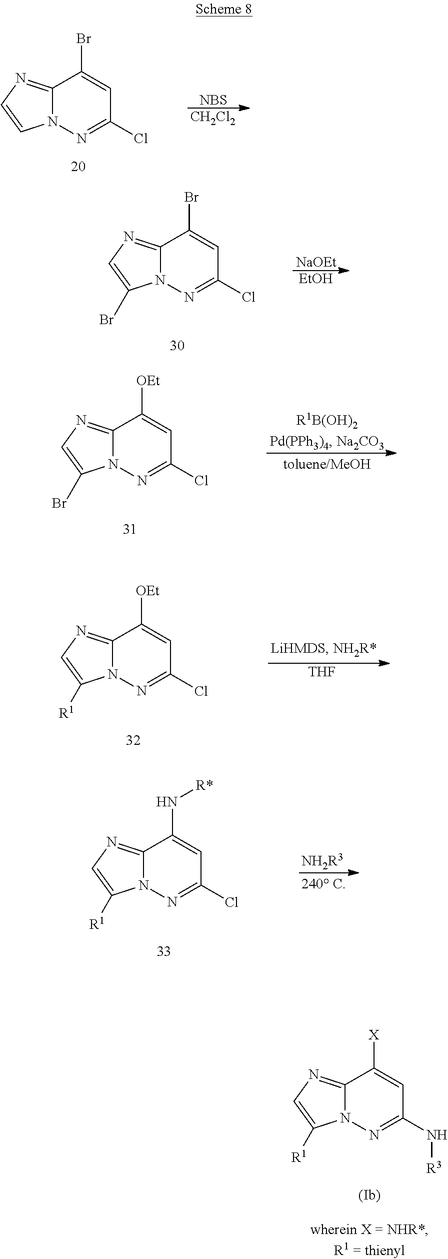

Compounds having formula (I) wherein R¹ is —C(O)NHR² are prepared according to the procedures outlined in Schemes 6-7 and are listed in Table 1.

TABLE 1
(I)

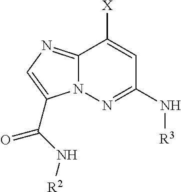

| Exam- | | | | |
|---|---|---|---|---|
| ple | X | R² | R³ | (M + H)⁺ |
| 1 | —NHPh | 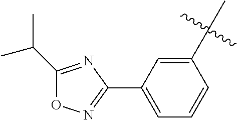 [SMILES: *c1cccc(-c2noc(C(C)C)n2)c1] | trans-4- aminocyclohexane | 552.3 |
| 2 | —NHPh | 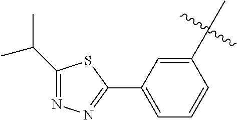 [SMILES: *c1cccc(-c2nnc(C(C)C)s2)c1] | trans-4- aminocyclohexane | 568.3 |
| 3 | —NHPh | 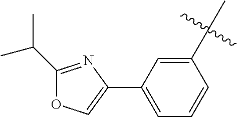 [SMILES: *c1cccc(-c2coc(C(C)C)n2)c1] | trans-4- aminocyclohexane | 551.4 |
| 4 | —NHPh | 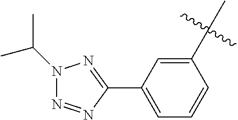 [SMILES: *c1cccc(-c2nnn(C(C)C)n2)c1] | trans-4- aminocyclohexane | 552.3 |
| 5 | —NHPh | 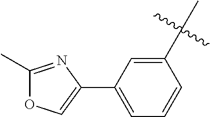 [SMILES: *c1cccc(-c2coc(C)n2)c1] | trans-4- aminocyclohexane | 523.3 |
| 6 | —NHPh | 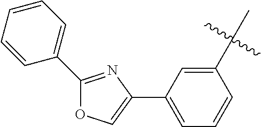 [SMILES: *c1cccc(-c2coc(-c3ccccc3)n2)c1] | trans-4- aminocyclohexane | 585.4 |
| 7 | —NHPh | 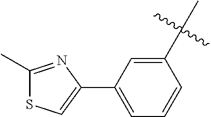 [SMILES: *c1cccc(-c2csc(C)n2)c1] | trans-4- aminocyclohexane | 539.3 |
| 8 | —NHMe | 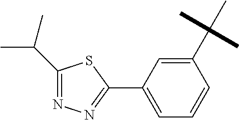 | trans-4- aminocyclohexane | 506.4 |
| 9 | —NHMe | 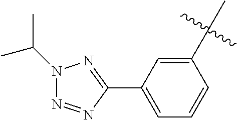 [SMILES: *c1cccc(-c2nnn(C(C)C)n2)c1] | trans-4- aminocyclohexane | 490.3 |
| 10 | —NHMe | 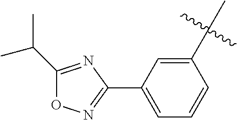 [SMILES: *c1cccc(-c2noc(C(C)C)n2)c1] | trans-4- aminocyclohexane | 490.3 |
| 11 | H | 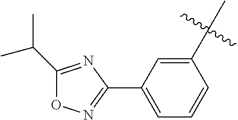 [SMILES: *c1cccc(-c2noc(C(C)C)n2)c1] | trans-4- aminocyclohexane | 461.2 |
| 12 | H | 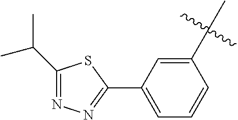 [SMILES: *c1cccc(-c2nnc(C(C)C)s2)c1] | trans-4- aminocyclohexane | 477.2 |
| 13 | H | 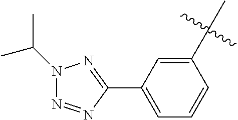 [SMILES: *c1cccc(-c2nnn(C(C)C)n2)c1] | trans-4- aminocyclohexane | 461.3 |
| 14 | H | 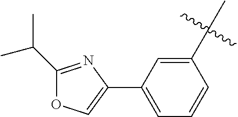 [SMILES: *c1cccc(-c2coc(C(C)C)n2)c1] | trans-4- aminocyclohexane | 460.3 |
| 15 | H | 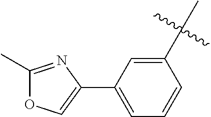 [SMILES: *c1cccc(-c2coc(C)n2)c1] | trans-4- aminocyclohexane | 432.4 |
| 16 | H | 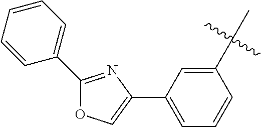 [SMILES: *c1cccc(-c2coc(-c3ccccc3)n2)c1] | trans-4- aminocyclohexane | 494.3 |

Compounds of formula (I) wherein R¹ is thienyl are prepared according to the procedures outlined in Scheme 8 and are listed in Table 2.

TABLE 2
(I)

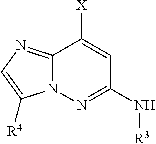

| Example | X | R⁴ | R³ | (M + H)⁺ |
|---|---|---|---|---|
| 17 | —NHPh | thiophen-3-yl | trans-4- | 405.3 |
| | | | aminocyclohexane | |
| 18 | —NH-2,4,6- | thiophen-3-yl | trans-4- | 447.3 |
| | tri-Me—Ph | | aminocyclohexane | |
| 19 | —NHPh | thiophen-3-yl | trans-4- | 433.3 |
| | | | dimethylamino- | |
| | | | cyclohexane | |
| 20 | —NHMe | thiophen-3-yl | trans-4- | 343.3 |
| | | | aminocyclohexane | |
| 21 | —NHcPr | thiophen-3-yl | trans-4- | 369.4 |
| | | | aminocyclohexane | |
| 22 | —NH-4- | thiophen-3-yl | trans-4- | 435.4 |
| | OMe—Ph | | aminocyclohexane | |
| 23 | —NH-(4-(5- | thiophen-3-yl | trans-4- | 531.4 |
| | isopropyl- | | aminocyclohexane | |
| | 1,3,4- | | | |
| | thiadiazol- | | | |
| | 2-yl)phenyl) | | | |

### DESCRIPTION OF THE SPECIFIC EMBODIMENTS

In the following examples, proton NMR spectra were recorded on either a Bruker 400 or 500 MHz NMR spectrometer. Chemical shifts are reported in δ values relative to tetramethylsilane. Liquid chromatography (LC)/mass spectra were run on a Shimadzu LC coupled to a Waters Micromass ZQ. HPLC retention times were obtained using one of the following two methods:

### Method A:

Waters analytical C18 sunfire column (4.6×150 mm, 3.5 μm); mobile phase: A=H₂O with 0.1% TFA, B=acetonitrile with 0.1% TFA; 1-15 min, 10% B→95% B; 15-18 min, 95% B; flow rate=1 mL/min; λ=254 nm; run time=18 min.

### Method B:

Waters analytical phenyl xbridge column (4.6×150 mm, 3.5 μm), mobile phase: A=H₂O with 0.1% TFA, B=acetonitrile with 0.1% TFA, 1-15 min, 10% B→95% B; 15-18 min, 95% B; flow rate=1 mL/min; λ=254 nm; run time=18 min.

Experimental procedures for the preparation of intermediates used in the synthesis of final products are shown below. The procedures below are representative procedures. One skilled in the art will appreciate that analogs with other alkyl or aryl groups at R⁵ (Schemes 1-5) may be prepared in a similar fashion.

### 3-(5-Isopropyl-1,2,4-oxadiazol-3-yl)aniline

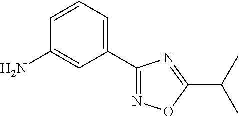

### Part A. (Z)—N′-hydroxy-3-nitrobenzimidamide

To a solution of hydroxylamine hydrochloride (62.2 g, 894 mmol) in pyridine (200 mL) at 0° C. was added 3-nitrobenzonitrile (22.08 g, 149 mmol). The reaction mixture was allowed to warm up to room temperature and was stirred overnight. The reaction mixture was diluted with ethyl acetate (1000 mL) and was washed with satd. aq NH₄Cl, satd. aq. NaHCO₃ and water. The organic layer was dried over MgSO₄ and concentrated to obtain crude (Z)—N-hydroxy-3-nitrobenzimidamide (32 g, 81% yield). The product was used without further purification. LCMS (ESI) m/e 182 [(M+H)⁺, calcd for C₇H₈N₃O₃ 182.1].

### Part B. 5-Isopropyl-3-(3-nitrophenyl)-1,2,4-oxadiazole

To a solution of isobutyryl chloride (4.13 g, 38.8 mmol) in dry pyridine (50 mL) at room temperature under nitrogen was added CDI (6.28 g, 38.8 mmol). The reaction mixture was stirred for 15 minutes and (Z)—N′-hydroxy-3-nitrobenzimidamide (5.4 g, 29.8 mmol) was added. The reaction mixture was heated in an oil bath at 110° C. for 4 hours. The mixture was concentrated to remove the pyridine. The residue was redissolved in ethyl acetate and was washed with water, brine, dried over MgSO₄, filtered, and concentrated. The residue was purified by column chromatography on silica gel (0%→60% ethyl acetate in hexanes) to afford 5-isopropyl-3-(3-nitrophenyl)-1,2,4-oxadiazole (4.91 g, 71% yield) as reddish oil: LCMS (ESI) m/e 234 [(M+H)⁺, calcd for C₁₁H₁₂N₃O₃ 234.1].

### Part C. 3-(5-Isopropyl-1,2,4-oxadiazol-3-yl)aniline

To a solution of 5-isopropyl-3-(3-nitrophenyl)-1,2,4-oxadiazole (4.91 g, 21.05 mmol) in absolute ethanol (50 mL) at room temperature was added ammonium chloride (13.51 g, 253 mmol). To the stirred suspension was added zinc dust (19.27 g, 295 mmol). The reaction mixture was stirred overnight at room temperature. No reaction was observed. The reaction mixture was then heated at reflux for 6 hours. Complete consumption of starting material was observed by LC-MS. The reaction mixture was filtered through a pad of diatomaceous earth (Celite®) and the filtrate was concentrated. The residue was dissolved in ethyl acetate (600 mL). The organic layer was washed with water, brine and dried over MgSO₄, filtered and concentrated to afford 3-(5-isopropyl-1,2,4-oxadiazol-3-yl)aniline (3.69 g, 86% yield). The product was used without further purification. LCMS (ESI) m/e 204 [(M+H)⁺, calcd for C₁₁H₁₂N₃O₃ 204.1].

### 3-(5-Isopropyl-1,3,4-thiadiazol-2-yl)aniline

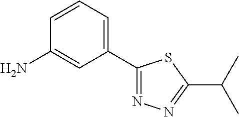

### Part A. N′-Isobutyryl-3-nitrobenzohydrazide

To a solution of isobutyric acid hydrazide (1.101 g, 10.78 mmol) in dichloromethane (80 mL) containing N,N-diisopropylethylamine (1.882 mL, 10.78 mmol) at room temperature under nitrogen was added 3-nitrobenzoyl chloride (2.00 g, 10.78 mmol). The reaction mixture was stirred for 30 minutes at room temperature. The product crashed out as a white solid. The reaction mixture was filtered through a Buchner funnel and the solid was washed with cold hexanes then dried under high vacuum overnight to afford N′-isobutyryl-3-nitrobenzohydrazide (2.09 g, 77% yield). LCMS (ESI) m/e 252 [(M+H)⁺, calcd for C₁₁H₁₄N₃O₄ 252.1].

### Part B. 2-Isopropyl-5-(3-nitrophenyl)-1,3,4-thiadiazole

To a solution of N′-isobutyryl-3-nitrobenzohydrazide (3.9 g, 15.52 mmol) in dry toluene (120 mL) at room temperature under nitrogen was added Lawesson's reagent (11.30 g, 27.9 mmol). The reaction mixture was heated at 100° C. overnight. Complete consumption of starting material was observed. The residue was purified by column chromatography on silica gel (0%→100% ethyl acetate in hexanes) to afford 2-isopropyl-5-(3-nitrophenyl)-1,3,4-thiadiazole (2.92 g, 75% yield). LCMS (ESI) m/e 250 [(M+H)⁺, calcd for C₁₁H₁₃N₃O₂S 250.1].

### Part C. 3-(5-Isopropyl-1,3,4-thiadiazol-2-yl)aniline

To a solution of the 2-isopropyl-5-(3-nitrophenyl)-1,3,4-thiadiazole (2.92 g, 11.71 mmol) in absolute ethanol (200 mL) at room temperature was added ammonium chloride (7.52 g, 141 mmol). To the stirred suspension was added zinc dust (10.72 g, 164 mmol). The reaction mixture was stirred overnight at room temperature. No reaction was observed. The reaction mixture was then heated to reflux for 6 hours. Complete consumption of starting material was observed by LC-MS. The reaction mixture was filtered through a pad of diatomaceous earth (Celite®) and the filtrate was concentrated. The residue was dissolved in ethyl acetate (600 mL). The organic layer was washed with water, brine, dried over MgSO₄, filtered and concentrated to afford 3-(5-isopropyl-1,3,4-thiadiazol-2-yl)aniline (2.39 g, 93% yield). The product was used without further purification. LCMS (ESI) m/e 220 [(M+H)⁺, calcd for C₁₁H₁₄N₃S 220.1].

### 3-(2-Isopropyloxazol-4-yl)aniline

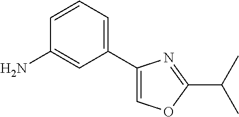

### Part A. 2-Isopropyl-4-(3-nitrophenyl)oxazole

2-Bromo-1-(3-nitrophenyl)ethanone (1.00 g, 4.10 mmol) and isobutyramide (0.821 g, 9.42 mmol) were heated in a sealed vessel to 140° C. for 3 hours. The material was cooled, diluted with diethyl ether and transferred to a separatory funnel containing water. The organic layer was washed with aq NaOH (0.5 M), aq HCl (0.5 M), brine, dried over Na₂SO₄, filtered, and concentrated to afford 2-isopropyl-4-(3-nitrophenyl)oxazole (0.8 g, 84% yield) as a yellow solid. LCMS (ESI) m/e 233.2 [(M+H)⁺, calcd for C₁₂H₁₃N₂O₃ 233.1].

### Part B. 3-(2-Isopropyloxazol-4-yl)aniline

To a mixture of 2-isopropyl-4-(3-nitrophenyl)oxazole (500 mg, 2.449 mmol) and ammonium chloride (1572 mg, 29.4 mmol) in ethanol (50 mL) was added zinc dust (2242 mg, 34.3 mmol). The mixture was heated at reflux for 3 h. The reaction was filtered through a pad of diatomaceous earth (Celite®). The filtrate was concentrated and redissolved in ethyl acetate. The organic layer was washed with water, brine, dried over Na₂SO₄, filtered, and concentrated to afford 3-(2-isopropyloxazol-4-yl)aniline (420 mg, 98% yield) as a brown oil, which was used without further purification. LCMS (ESI) m/e 175.2 [(M+H)⁺, calcd for C₁₀H₁₁N₂O 175.1].

### 3-(2-Isopropyl-2H-tetrazol-5-yl)aniline

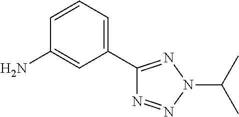

### Part A. 5-(3-Nitrophenyl)-2H-tetrazole

A mixture of 3-nitrobenzonitrile (2.00 g, 13.50 mmol), sodium azide (5.27 g, 81 mmol) and ammonia hydrochloride (4.33 g, 81 mmol) in DMF (30 mL) was heated at reflux for 16 h. The mixture was cooled and then poured into (200 mL) 1N HCl and diluted with water (100 mL). The precipitate that formed was collected to afford 5-(3-nitrophenyl)-2H-tetrazole (2.5 g, 97% yield) as a colorless solid, which was used directly in the next step.

### Part B. 2-Isopropyl-5-(3-nitrophenyl)-2H-tetrazole

A mixture of 5-(3-nitrophenyl)-2H-tetrazole (200 mg, 1.046 mmol), 2-iodopropane (0.125 mL, 1.256 mmol) and potassium carbonate (318 mg, 2.302 mmol) in DMF (20 mL) was heated to 100° C. in a sealed tube for 12 h. The mixture was transferred to a separatory funnel containing ethyl acetate. The organic layer was washed with brine, dried over Na₂SO₄, filtered, and concentrated to afford 2-isopropyl-5-(3-nitrophenyl)-2H-tetrazole (200 mg, 82% yield) as a yellow solid. LCMS (ESI) m/e 234.2 [(M+H)⁺, calcd for C₁₀H₁₂N₅O₂ 234.1]

### Part C. 3-(2-Isopropyl-2H-tetrazol-5-yl)aniline

To a mixture of 2-isopropyl-5-(3-nitrophenyl)-2H-tetrazole (200 mg, 0.858 mmol) and ammonium chloride (550 mg, 10.29 mmol) in ethanol (10 mL) was added zinc dust (785 mg, 12.01 mmol). The mixture was heated at reflux for 3 h. The mixture was filtered through a pad of diatomaceous earth (Celite®). The filtrate was concentrated and redissolved in ethyl acetate. The organic layer was washed with water, brine, dried over Na₂SO₄, filtered, and concentrated to afford 3-(2-isopropyl-2H-tetrazol-5-yl)aniline (170 mg, 98% yield) as a brown oil, which was used without further purification. LCMS (ESI) m/e 204.3 [(M+H)⁺, calcd for C₁₀H₁₄N₅ 204.1].

### 3-(2-Methylthiazol-5-yl)aniline

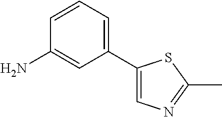

### Part A. 2-Methyl-4-(3-nitrophenyl)thiazole

2-bromo-1-(3-nitrophenyl)ethanone (2.00 g, 8.20 mmol), ethanethioamide (0.616 g, 8.20 mmol) and ethanol (50 mL) were combined and heated at reflux for 2 h. The reaction mixture was cooled to room temperature and concentrated. The product was tritrated with hexane containing a small amount of ethyl acetate. The suspension was cooled to 0° C. and the solid was collected on a Buchner funnel to give 2-methyl-4-(3-nitrophenyl)thiazole (1.78 g, 99% yield) as a colorless solid: ¹H NMR (400 MHz, DMSO-d₆) δ 8.73 (t, J=1.9 Hz, 1H), 8.39 (d, J=7.8 Hz, 1H), 8.27 (s, 1H), 8.18 (dd, J=8.1, 2.3 Hz, 1H), 7.73 (t, J=8.1 Hz, 1H), 2.75 (s, 3H); LCMS (ESI) m/e 221.2 [(M+H)⁺, calcd for C₁₀H₉N₂O₂S 221.0].

### Part B. 3-(2-Methylthiazol-5-yl)aniline

To a suspension of 2-methyl-4-(3-nitrophenyl)thiazole (1.65 g, 7.49 mmol) in ethanol (100 mL) was added ammonium chloride (4.81 g, 90 mmol) and zinc dust (6.86 g, 105 mmol). The reaction mixture was heated at reflux for 2.5 h. The mixture was cooled to room temperature and was filtered through a pad of diatomaceous earth (Celite®) and the filtrate was concentrated. The reaction mixture was transferred to a separatory funnel containing saturated aqueous NaHCO₃ solution (25 mL). The aqueous layer was extracted with ethyl acetate (3×75 mL). The combined organic layers were washed with brine (25 mL), dried over MgSO₄, filtered and concentrated. The residue was purified via column chromatography on silica gel (40%→50% ethyl acetate in hexane) to afford 3-(2-methylthiazol-4-yl)aniline (900 mg, 63% yield) as an off-white solid: ¹H NMR (400 MHz, DMSO-d₆) δ 7.69 (s, 1H), 7.19 (d, J=1.0 Hz, 1H), 7.05 (d, J=5.0 Hz, 2H), 6.47-6.56 (m, 1H), 5.14 (s, 2H), 2.69 (s, 3H); LCMS (ESI) m/e 191.25 [(M+H)⁺, calcd for C₁₀H₁₁N₂S 191.06].

Experimental procedures for the preparation of final products (compounds of formula (Ia) and (Ib)) are described below.

### Example 1

### 6-(trans-4-Aminocyclohexylamino)-N-(3-(5-isopropyl-1,2,4-oxadiazol-3-yl)phenyl)-8-(phenylamino)imidazo[1,2-b]pyridazine-3-carboxamide

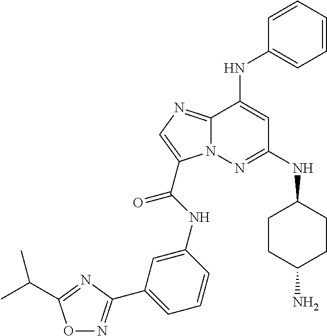 [SMILES: CC(C)c1nc(-c2cccc(NC(=O)c3cnc4c(Nc5ccccc5)cc(N[C@H]5CC[C@H](N)CC5)nn34)c2)no1]

### Part A. N-(4-Methoxybenzyl)-N-phenylimidazo[1,2-b]pyridazin-8-amine

To a solution of 8-bromo-6-chloroimidazo[1,2-b]pyridazine (340 mg, 1.463 mmol) (prepared as described by Vaccaro W. et al. United States Patent Appl. US 2008/0045536 A1, 2008) and N-(4-methoxybenzyl)aniline (312 mg, 1.46 mmol) in THF (5 mL) at 0° C. was added LiHMDS (4.39 mL, 4.39 mmol, 1 M in THF). The reaction mixture was stirred at 0° C. for 3 h. The starting material was still present. Additional LiHMDS (4.39 mL, 4.39 mmol, 1 M in THF) was added and the reaction was stirred at room temperature for 2 h. LCMS indicated complete consumption of the starting material. The reaction mixture was transferred to a separatory funnel containing saturated aqueous NaHCO₃ solution (25 mL) and the aqueous layer was extracted with EtOAc (3×25 mL). The combined organic layers were concentrated and the residue was purified by column chromatography on silica gel (10%→30% ethyl acetate in hexanes) to afford 6-chloro-N-(4-methoxybenzyl)-N-phenylimidazo[1,2-b]pyridazin-8-amine (280 mg, 53% yield) as a yellow solid: LRMS (ESI) m/e 365.2 [(M+H)⁺, calcd for C₂₀H₁₈N₄OCl 365.2].

### Part B. 6-Chloro-8-((4-methoxybenzyl)(phenyl)amino)imidazo[1,2-b]pyridazine-3-carboxylic acid

A solution of 6-chloro-N-(4-methoxybenzyl)-N-phenylimidazo[1,2-b]pyridazin-8-amine (0.500 g, 1.37 mmol) in THF (20 mL) was cooled to −78° C. To this solution, was added slowly n-BuLi (1.3 mL, 2.1 mmol, 1.6 M in hexanes). The solution was stirred at at −78° C. for 30 minutes. During this time the solution turned red. Dry ice (0.603 g, 13.7 mmol) was added to the reaction mixture and it was allowed to warm to room temperature. LCMS indicated complete consumption of the starting material. The solution was concentrated in vacuo. The residue was diluted with ethyl acetate (50 mL) and was washed with saturated aqueous NaHCO₃ solution (20 mL). The organic layer was dried over Na₂SO₄, filtered, and concentrated in vacuo to afford 6-chloro-8-((4-methoxybenzyl)(phenyl)amino)imidazo[1,2-b]pyridazine-3-carboxylic acid (560 mg, 100% yield) as a yellow solid: ¹H NMR (400 MHz, DMSO-d₆) δ 7.67 (s, 1H), 7.44 (t, J=7.7 Hz, 2H), 7.32 (t, J=7.4 Hz, 1H), 7.23 (d, J=7.3 Hz, 2H), 7.16 (d, J=8.6 Hz, 2H), 6.81 (d, J=8.6 Hz, 2H), 5.90 (s, 2H), 5.61 (s, 1H), 3.68 (s, 3H); LRMS (ESI) m/e 409.3 [(M+H)⁺, calcd for C₂₁H₁₈N₄ClO₃ 409.1].

### Part C. 6-Chloro-N-(3-(5-isopropyl-1,2,4-oxadiazol-3-yl)phenyl)-8-((4-methoxybenzyl)(phenyl)amino)imidazo[1,2-b]pyridazine-3-carboxamide

A mixture of 6-chloro-8-((4-methoxybenzyl)(phenyl)amino)imidazo[1,2-b]pyridazine-3-carboxylic acid (150 mg, 0.367 mmol) and 3-(5-isopropyl-1,2,4-oxadiazol-3-yl)aniline (149 mg, 0.734 mmol) (prepared as described above) was dissolved in CH₂Cl₂ (5 mL) and cooled to 0° C. To this was added N,N-diisopropylethyl amine (0.320 mL, 1.83 mmol) and O-benzotriazol-1-yl-N,N,N′,N′-tetramethyluronium tetrafluoroborate (TBTU) (236 mg, 0.734 mmol). The resulting solution was stirred at room temperature for 12 h. The mixture was transferred to a reparatory funnel containing saturated aqueous NaHCO₃ solution (20 mL) and the aqueous layer was extracted with ethyl acetate (3×25 mL). The organic layer was concentrated and the residue was purified by column chromatography on silica gel (20%→50% ethyl acetate in hexanes) to afford 6-chloro-N-(3-(5-isopropyl-1,2,4-oxadiazol-3-yl)phenyl)-8-((4-methoxybenzyl)(phenyl)amino)imidazo[1,2-b]pyridazine-3-carboxamide (96 mg, 44% yield) as a yellow solid: LRMS (ESI) m/e 594.2 [(M+H)⁺, calcd for C₃₂H₂₉N₇O₃Cl 594.2].

### Part D. 6-(trans-4-Aminocyclohexylamino)-N-(3-(5-isopropyl-1,2,4-oxadiazol-3-yl)phenyl)-8-((4-methoxybenzyl)(phenyl)amino)imidazo[1,2-b]pyridazine-3-carboxamide

6-Chloro-N-(3-(5-isopropyl-1,2,4-oxadiazol-3-yl)phenyl)-8-((4-methoxybenzyl)(phenyl)amino)imidazo[1,2-b]pyridazine-3-carboxamide (85 mg, 0.143 mmol) and cyclohexane-1,4-diamine (98 mg, 0.858 mmol) were heated in a microwave at 220° C. for 2 hours. The mixture was transferred to a separatory funnel containing ethyl acetate and the organic layer was washed with water. The aqueous layer was extracted with ethyl acetate (3×20 mL). The organic layer was concentrated and the residue was purified by column chromatography on silica gel (10% MeOH in CH₂Cl₂) to afford 6-(trans-4-aminocyclohexylamino)-N-(3-(5-isopropyl-1,2,4-oxadiazol-3-yl)phenyl)-8-((4-methoxybenzyl)(phenyl)amino)imidazo[1,2-b]pyridazine-3-carboxamide (60 mg, 62% yield) as a colorless solid: ¹H NMR (400 MHz, CD₃OD) δ 8.26 (t, J=1.6 Hz, 1H), 7.96 (s, 1H), 7.93 (dd, J=8.3, 1.0 Hz, 1H), 7.78 (d, J=7.8 Hz, 1H), 7.50 (t, J=7.9 Hz, 1H), 7.34 (t, J=7.7 Hz, 2H), 7.22 (t, J=7.4 Hz, 1H), 7.15 (d, J=8.6 Hz, 2H), 7.11 (d, J=7.3 Hz, 2H), 6.74 (d, J=8.6 Hz, 2H), 5.63 (s, 2H), 5.50 (s, 1H), 3.69 (s, 3H), 3.54-3.64 (m, 1H), 2.56-2.64 (m, 1H), 2.14 (d, J=12.1 Hz, 2H), 1.87 (d, J=11.6 Hz, 2H), 1.42 (d, J=6.8 Hz, 6H), 1.18-1.38 (m, 4H); LRMS (ESI) m/e 672.3 [(M+H)⁺, calcd for C₃₈H₄₂N₉O₃ 672.3].

### Part E. 6-(trans-4-Aminocyclohexylamino)-N-(3-(5-isopropyl-1,2,4-oxadiazol-3-yl)phenyl)-8-(phenylamino)imidazo[1,2-b]pyridazine-3-carboxamide

6-(trans-4-Aminocyclohexylamino)-N-(3-(5-isopropyl-1,2,4-oxadiazol-3-yl)phenyl)-8-((4-methoxybenzyl)(phenyl)amino)imidazo[1,2-b]pyridazine-3-carboxamide (80 mg, 0.119 mmol) was dissolved in CH₂Cl₂ (1 mL) and was cooled to 0° C. To this cooled solution was added TFA (0.5 mL, 6.49 mmol). The reaction mixture was stirred at 0° C. for 1 hour. The reaction mixture was transferred to a separatory funnel containing saturated aqueous NaHCO₃ solution (10 mL). The aqueous layer was extracted with ethyl acetate (3×20 mL). The organic layer was concentrated and the residue was purified by column chromatography on silica gel (10% MeOH with NH₃ (2M) in CH₂Cl₂) to afford 6-(trans-4-aminocyclohexylamino)-N-(3-(5-isopropyl-1,2,4-oxadiazol-3-yl)phenyl)-8-(phenylamino)imidazo[1,2-b]pyridazine-3-carboxamide (56 mg, 85% yield) as an off-white solid: ¹H NMR (400 MHz, DMSO-d₆) δ 11.16 (s, 1H), 9.29 (s, 1H), 8.73 (s, 1H), 8.04 (s, 1H), 7.83 (d, J=1.1 Hz, 1H); 7.81 (br s, 3H), 7.67 (t, J=7.9 Hz, 1H), 7.53 (d, J=9.1 Hz, 1H), 7.39-7.48 (m, 4H), 7.16-7.22 (m, 1H), 6.97 (d, J=6.5 Hz, 1H), 6.29 (s, 1H), 3.67 (br s, 1H), 3.38 (quint, J=7.0 Hz, 1H), 3.06 (br s, 1H), 2.21 (d, J=13.1 Hz, 2H), 1.98 (d, J=10.8 Hz, 2H), 1.41 (d, J=6.8 Hz, 6H), 1.22-1.49 (m, 4H); LRMS (ESI) m/e 552.3 [(M+H)⁺, calcd for C₃₀H₃₄N₉O₂ 552.3]. HPLC retention time (method A): t_{R}=10.76 min; HPLC retention time (method B): t_{R}=10.83 min.

### Example 2

### 6-(trans-4-Aminocyclohexylamino)-N-(3-(5-isopropyl-1,3,4-thiadiazol-2-yl)phenyl)-8-(phenylamino)imidazo[1,2-b]pyridazine-3-carboxamide

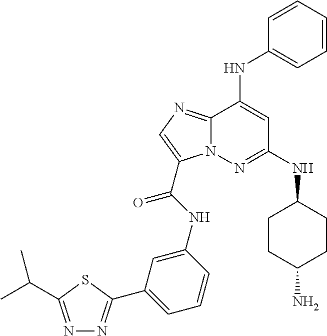 [SMILES: CC(C)c1nnc(-c2cccc(NC(=O)c3cnc4c(Nc5ccccc5)cc(N[C@H]5CC[C@H](N)CC5)nn34)c2)s1]

Prepared by the method described in Example 1 using 3-(5-isopropyl-1,3,4-thiadiazol-2-yl)aniline in Part C followed by purification by HPLC (acetonitrile with 0.1% TFA/water with 0.1% TFA) to give 6-(trans-4-aminocyclohexylamino)-N-(3-(5-isopropyl-1,3,4-thiadiazol-2-yl)phenyl)-8-(phenylamino)imidazo[1,2-b]pyridazine-3-carboxamide (12 mg) as a TFA salt: ¹H NMR (400 MHz, DMSO-d₆) δ 11.16 (s, 1H), 9.29 (s, 1H), 8.56 (s, 1H), 8.05 (s, 1H), 7.85 (d, J=4.5 Hz, 3H), 7.70-7.75 (m, 2H), 7.62-7.67 (m, 1H), 7.40-7.47 (m, 4H), 7.17-7.21 (m, 1H), 6.96 (d, J=7.3 Hz, 1H), 6.28 (s, 1H), 3.72 (br s, 1H), 3.52 (ddd, J=13.7, 7.1, 6.9 Hz, 1H), 3.11 (br s, 1H), 2.20 (d, J=11.1 Hz, 2H), 2.00 (d, J=11.3 Hz, 2H), 1.52-1.63 (m, 2H), 1.43 (d, J=6.8 Hz, 6H), 1.26-1.38 (m, 2H); LRMS (ESI) m/e 568.3 [(M+H)⁺, calcd for C₃₀H₃₄N₉OS 568.3]. HPLC retention time (method A): t_{R}=11.48 min; HPLC retention time (method B): t_{R}=11.70 min.

### Example 3

### 6-(trans-4-Aminocyclohexylamino)-N-(3-(2-isopropyloxazol-4-yl)phenyl)-8-(phenylamino)imidazo[1,2-b]pyridazine-3-carboxamide

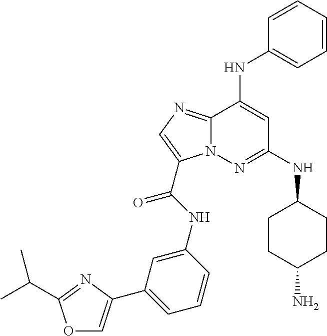 [SMILES: CC(C)c1nc(-c2cccc(NC(=O)c3cnc4c(Nc5ccccc5)cc(N[C@H]5CC[C@H](N)CC5)nn34)c2)co1]

Prepared by the method described in Example 1 using 3-(2-isopropyloxazol-4-yl)aniline in Part C followed by purification by HPLC (acetonitrile with 0.1% TFA/water with 0.1% TFA) to give 6-(trans-4-aminocyclohexylamino)-N-(3-(2-isopropyloxazol-4-yl)phenyl)-8-(phenylamino)imidazo[1,2-b]pyridazine-3-carboxamide (8 mg) as a TFA salt: ¹H NMR (400 MHz, DMSO-d₆) δ 11.04 (s, 1H), 9.29 (s, 1H), 8.51 (s, 1H), 8.46 (s, 1H), 8.02 (s, 1H), 7.80 (br s, 3H), 7.55-7.60 (m, 1H), 7.51 (t, J=7.8 Hz, 1H), 7.39-7.47 (m, 4H), 7.16-7.24 (m, 2H), 6.95 (d, J=6.5 Hz, 1H), 6.28 (s, 1H), 3.65 (s br 1H), 3.15 (quin, J=7.0 Hz, 1H), 3.06 (s br, 1H), 2.20 (d, J=12.3 Hz, 2H), 1.97 (d, J=13.3 Hz, 2H), 1.33 (d, J=7.1 Hz, 6H), 1.26-1.46 (m, 4H); LRMS (ESI) m/e 551.4 [(M+H)⁺, calcd for C₃₁H₃₅N₈O₂ 551.3]. HPLC retention time (method A): t_{R}=10.43 min; HPLC retention time (method B): t_{R}=10.68 min.

### Example 4

### 6-(trans-4-Aminocyclohexylamino)-N-(3-(2-isopropyl-2H-tetrazol-5-yl)phenyl)-8-(phenylamino)imidazo[1,2-b]pyridazine-3-carboxamide

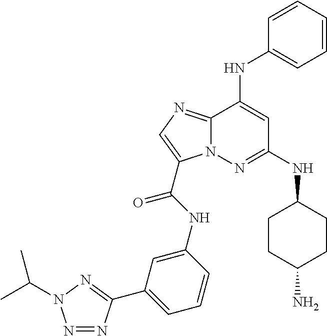 [SMILES: CC(C)n1nnc(-c2cccc(NC(=O)c3cnc4c(Nc5ccccc5)cc(N[C@H]5CC[C@H](N)CC5)nn34)c2)n1]

Prepared by the method described in Example 1 using 3-(2-isopropyl-2H-tetrazol-5-yl)aniline in Part C followed by purification by HPLC (acetonitrile with 0.1% TFA/water with 0.1% TFA) to give 6-(trans-4-aminocyclohexylamino)-N-(3-(2-isopropyl-2H-tetrazol-5-yl)phenyl)-8-(phenylamino)imidazo[1,2-b]pyridazine-3-m carboxamide (30 mg) as a TFA salt: ¹H NMR (400 MHz, CD₃OD) δ 8.44 (t, J=1.5 Hz, 1H), 8.07 (s, 1H), 7.86 (t, J=9.8 Hz, 2H), 7.55 (t, J=7.9 Hz, 1H), 7.40-7.46 (m, 2H), 7.33-7.38 (m, 2H), 7.20 (t, J=7.3 Hz, 1H), 6.26 (s, 1H), 5.17 (quin, J=6.7 Hz, 1H), 3.76-3.85 (m, 1H), 3.12-3.20 (m, 1H), 2.36 (d, J=11.6 Hz, 2H), 2.12 (d, J=12.1 Hz, 2H), 1.70 (d, J=6.5 Hz, 6H), 1.65-1.77 (m, 2H), 1.37-1.49 (m, 2H); LRMS (ESI) m/e 552.3 [(M+H)⁺, calcd for C₂₉H₃₄N₁₁O 552.3]. HPLC retention time (method A): t_{R}=10.71 min; HPLC retention time (method B): t_{R}=10.68 min.

### Example 5

### 6-(trans-4-Aminocyclohexylamino)-N-(3-(2-methyloxazol-4-yl)phenyl)-8-(phenylamino)imidazo[1,2-b]pyridazine-3-carboxamide

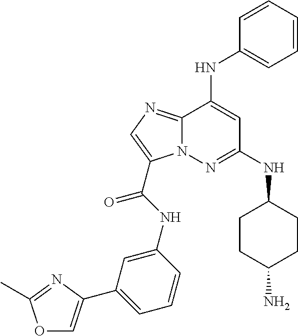 [SMILES: Cc1nc(-c2cccc(NC(=O)c3cnc4c(Nc5ccccc5)cc(N[C@H]5CC[C@H](N)CC5)nn34)c2)co1]

Prepared by the method described in Example 1 using 3-(2-methyloxazol-4-yl)aniline in Part C to give 6-(trans-4-aminocyclohexylamino)-N-(3-(2-methyloxazol-4-yl)phenyl)-8-(phenylamino)imidazo[1,2-b]pyridazine-3-carboxamide (18 mg) as a yellow solid: ¹H NMR (500 MHz, DMSO-d₆) δ 11.18 (s, 1H), 8.50 (s, 1H), 8.08 (br s, 1H), 8.02 (s, 1H), 7.72 (d, J=7.3 Hz, 1H), 7.50-7.54 (m, 1H), 7.40-7.49 (m, 4H), 7.18 (t, J=5.5 Hz, 1H), 6.92 (d, J=7.0 Hz, 1H), 6.30 (s, 1H), 3.68 (br s, 1H), 2.51 (s, 3H), 2.12 (d, J=8.2 Hz, 2H), 1.76 (d, J=9.2 Hz, 2H), 1.15-1.29 (m, 4H); LRMS (ESI) m/e 523.3 [(M+H)⁺, calcd for C₂₉H₃₁N₈O₂ 523.3]. HPLC retention time (method A): t_{R}=9.70 min; HPLC retention time (method B): t_{R}=9.85 min.

### Example 6

### 6-(trans-4-Aminocyclohexylamino)-8-(phenylamino)-N-(3-(2-phenyloxazol-4-yl)phenyl)imidazo[1,2-b]pyridazine-3-carboxamide

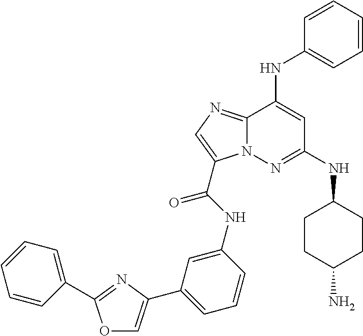 [SMILES: N[C@H]1CC[C@H](Nc2cc(Nc3ccccc3)c3ncc(C(=O)Nc4cccc(-c5coc(-c6ccccc6)n5)c4)n3n2)CC1]

Prepared by the method described in Example 1 using 3-(2-phenyloxazol-4-yl)aniline in Part C followed by purification by HPLC (acetonitrile with 0.1% TFA/water with 0.1% TFA) to give 6-(trans-4-aminocyclohexylamino)-8-(phenylamino)-N-(3-(2-phenyloxazol-4-yl)phenyl)imidazo[1,2-b]pyridazine-3-carboxamide (15 mg) as a TFA salt: ¹H NMR (400 MHz, DMSO-d₆) δ 11.10 (s, 1H), 9.29 (s, 1H), 8.77 (s, 1H), 8.55 (s, 1H), 8.06-8.10 (m, 2H), 8.04 (s, 1H), 7.81 (d, J=4.8 Hz, 3H), 7.69 (d, J=7.8 Hz, 1H), 7.56-7.61 (m, 4H), 7.40-7.47 (m, 4H), 7.32 (d, J=7.6 Hz, 1H), 7.17-7.21 (m, 1H), 6.97 (d, J=7.1 Hz, 1H), 6.30 (s, 1H), 3.67 (br s, 1H), 3.04 (br s, 1H), 2.22 (d, J=11.8 Hz, 2H), 1.96 (d, J=9.1 Hz, 2H), 1.29-1.47 (m, 4H); LRMS (ESI) m/e 585.4 [(M+H)⁺, calcd for C₃₄H₃₃N₈O₂ 585.3]. HPLC retention time (method A): t_{R}=11.47 min; HPLC retention time (method B): t_{R}=11.53 min.

### Example 7

### 6-(trans-4-Aminocyclohexylamino)-N-(3-(2-methylthiazol-4-yl)phenyl)-8-(phenylamino)imidazo[1,2-b]pyridazine-3-carboxamide

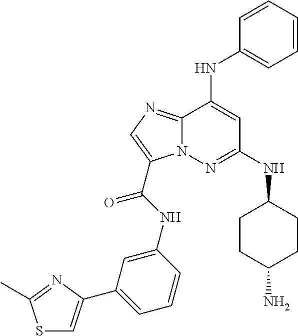 [SMILES: Cc1nc(-c2cccc(NC(=O)c3cnc4c(Nc5ccccc5)cc(N[C@H]5CC[C@H](N)CC5)nn34)c2)cs1]

Prepared by the method described in Example 1 using 3-(2-methylthiazol-4-yl)aniline in Part C followed by purification by HPLC (acetonitrile with 0.1% TFA/water with 0.1% TFA) to give 6-(trans-4-aminocyclohexylamino)-N-(3-(2-methylthiazol-4-yl)phenyl)-8-(phenylamino)imidazo[1,2-b]pyridazine-3-carboxamide (8 mg) as a TFA salt: ¹H NMR (400 MHz, CD₃OD) δ 8.41 (br s, 1H), 8.08 (s, 1H), 7.70 (d, J=7.8 Hz, 1H), 7.66 (s, 1H), 7.40-7.49 (m, 3H), 7.33-7.37 (m, 3H), 7.20 (t, J=7.4 Hz, 1H), 6.27 (s, 1H), 3.71 (br s, 1H), 3.11 (br s, 1H), 2.75 (s, 3H), 2.35 (d, J=8.6 Hz, 2H), 2.07 (d, J=11.1 Hz, 2H), 1.39-1.50 (m, 4H); LRMS (ESI) m/e 539.3 [(M+H)⁺, calcd for C₂₉H₃₁N₈OS 539.2]. HPLC retention time (method A): t_{R}=9.63 min; HPLC retention time (method B): t_{R}=9.94 min.

### Example 8

### 6-(trans-4-Aminocyclohexylamino)-N-(3-(5-isopropyl-1,3,4-thiadiazol-2-yl)phenyl)-8-(methylamino)imidazo[1,2-b]pyridazine-3-carboxamide

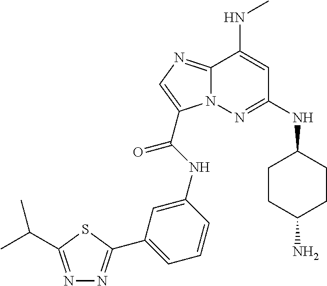 [SMILES: CNc1cc(N[C@H]2CC[C@H](N)CC2)nn2c(C(=O)Nc3cccc(-c4nnc(C(C)C)s4)c3)cnc12]

Prepared by the method described in Example 1 using 1-(4-methoxyphenyl)-N-methylmethanamine in Part A and 3-(5-isopropyl-1,3,4-thiadiazol-2-yl)aniline in Part C followed by purification by HPLC (acetonitrile with 0.1% TFA/water with 0.1% TFA) to give 6-(trans-4-aminocyclohexylamino)-N-(3-(5-isopropyl-1,3,4-thiadiazol-2-yl)phenyl)-8-(methylamino)imidazo[1,2-b]pyridazine-3-carboxamide (6 mg) as a TFA salt: ¹H NMR (400 MHz, CD₃OD) δ 11.24 (s, 1H), 8.40-8.44 (m, 1H), 8.02 (s, 1H) 7.98 (s, 1H), 7.50-7.48 (m, 2H), 5.61 (s, 1H), 3.87-3.95 (m, 1H), 3.52 (dt, J=13.8, 6.8 Hz, 1H), 3.34 (dd, J=3.3, 1.5 Hz, 1H), 2.94 (s, 3H), 2.36 (d, J=11.8 Hz, 2H), 2.10-2.17 (m, 4H), 1.49 (d, J=6.8 Hz, 6H), 1.41-1.48 (m, 2H); LRMS (ESI) m/e 506.4 [(M+H)⁺, calcd for C₂₅H₃₂N₉OS 506.2]. HPLC retention time (method A): t_{R}=9.47 min; HPLC retention time (method B): t_{R}=9.40 min.

### Example 9

### 6-(trans-4-Aminocyclohexylamino)-N-(3-(2-isopropyl-2H-tetrazol-5-yl)phenyl)-8-(methylamino)imidazo[1,2-b]pyridazine-3-carboxamide

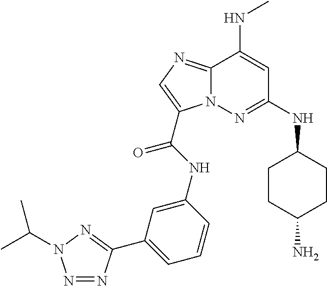 [SMILES: CNc1cc(N[C@H]2CC[C@H](N)CC2)nn2c(C(=O)Nc3cccc(-c4nnn(C(C)C)n4)c3)cnc12]

Prepared by the method described in Example 1 using 1-(4-methoxyphenyl)-N-methylmethanamine in Part A and 3-(2-isopropyl-2H-tetrazol-5-yl)aniline in Part C to give 6-(trans-4-aminocyclohexylamino)-N-(3-(2-isopropyl-2H-tetrazol-5-yl)phenyl)-8-(methylamino)imidazo[1,2-b]pyridazine-3-carboxamide (23 mg) as a yellow solid: ¹H NMR (400 MHz, DMSO-d₆) δ 11.29 (s, 1H), 8.59 (br s, 1H), 7.93 (s, 1H), 7.84 (d, J=7.6 Hz, 1H), 7.67-7.74 (m, 1H), 7.63 (t, J=7.8 Hz, 1H), 7.30 (d, J=4.3 Hz, 1H), 6.82 (d, J=7.3 Hz, 1H), 5.60 (s, 1H), 5.20 (ddd, J=13.2, 6.5, 6.3 Hz, 1H), 3.68 (br s, 1H), 2.83 (d, J=4.3 Hz, 3H), 2.73 (br s, 1H), 2.15 (br s, 2H), 1.86 (br s, 2H), 1.64 (d, J=6.5 Hz, 6H), 1.23-1.38 (m, 4H); LRMS (ESI) m/e 490.3 [(M+H)⁺, calcd for C₂₄H₃₂N₁₁O 490.3]. HPLC retention time (method A): t_{R}=10.71 min; HPLC retention time (method B): t_{R}=10.68 min.

### Example 10

### 6-(trans-4-Aminocyclohexylamino)-N-(3-(5-isopropyl-1,2,4-oxadiazol-3-yl)phenyl)-8-(methylamino)imidazo[1,2-b]pyridazine-3-carboxamide

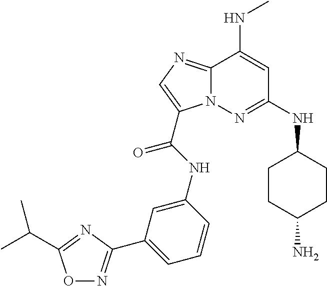 [SMILES: CNc1cc(N[C@H]2CC[C@H](N)CC2)nn2c(C(=O)Nc3cccc(-c4noc(C(C)C)n4)c3)cnc12]

Prepared by the method described in Example 1 using 1-(4-methoxyphenyl)-N-methylmethanamine in Part A to give 6-(trans-4-aminocyclohexylamino)-N-(3-(5-isopropyl-1,2,4-oxadiazol-3-yl)phenyl)-8-(methylamino)imidazo[1,2-b]pyridazine-3-carboxamide (99 mg) as a tan solid: ¹H NMR (400 MHz, DMSO-d₆) δ 11.23 (s, 1H), 8.72 (s, 1H), 7.95 (s, 1H), 7.85 (d, J=3.5 Hz, 2H), 7.81 (d, J=7.8 Hz, 1H), 7.67 (t, J=7.8 Hz, 1H), 7.53 (d, J=8.8 Hz, 1H), 7.35 (d, J=3.8 Hz, 1H), 6.85 (br s, 1H), 5.62 (s, 1H), 3.68 (br s, 1H), 3.37 (ddd, J=14.0, 6.9, 6.8 Hz, 1H), 3.08 (br s, 1H), 2.83 (d, J=2.5 Hz, 3H), 2.21 (d, J=12.6 Hz, 2H), 1.99 (d, J=11.1 Hz, 2H), 1.40 (d, J=7.1 Hz, 6H), 1.27-1.52 (m, 4H); LRMS (ESI) m/e 490.3 [(M+H)⁺, calcd for C₂₅H₃₂N₉O₂ 490.3]. HPLC retention time (method A): t_{R}=10.78 min; HPLC retention time (method B): t_{R}=10.97 min.

### Example 11

### 6-(trans-4-Aminocyclohexylamino)-N-(3-(5-isopropyl-1,2,4-oxadiazol-3-yl)phenyl)imidazo[1,2-b]pyridazine-3-carboxamide

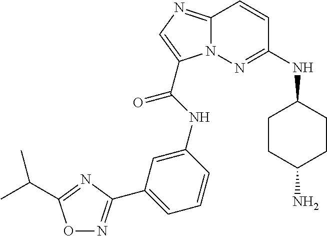 [SMILES: CC(C)c1nc(-c2cccc(NC(=O)c3cnc4ccc(N[C@H]5CC[C@H](N)CC5)nn34)c2)no1]

### Part A. Ethyl 2-chloro-3-oxopropanoate (26)

A mixture of ethyl formate (3.3 mL, 41 mmol) and ethyl 2-chloroacetate (3.5 mL, 41 mmol) in THF (80 mL) was cooled to −78° C. To this mixture was added potassium t-butoxide (81 mL, 81 mmol) slowly such that the temperature of the reaction stayed below −68° C. After the addition was complete, the reaction mixture was stirred at −78° C. for 1 h and was then warmed to 0° C. and was stirred for an additional 3 h. The reaction mixture was quenched at 0° C. with 1 N HCl (30 mL), and then cautiously acidified to pH 4 with conc. HCl (ca. 5 mL). The mixture was transferred to a reparatory funnel containing water (30 mL). Solid sodium chloride was added and the aqueous layer was extracted with ether (3×150 mL). The combined organic layers were washed with brine, dried over Na₂SO₄, filtered, concentrated and placed under vacuum to afford ethyl 2-chloro-3-oxopropanoate (4.3 g, 71% yield) as a pale-yellow oil which was directly in the next step.

### Part B. Ethyl 6-chloroimidazo[1,2-b]pyridazine-3-carboxylate

A suspension of ethyl 2-chloro-3-oxopropanoate (2.00 g, 13.28 mmol) and 6-chloropyridazin-3-amine (1.721 g, 13.28 mmol) in EtOH (30 mL) in a 350 mL pressure vessel was heated at 90° C. for 14 h. The mixture was cooled to room temperature and was concentrated. The residue was taken up in ethyl acetate/ethanol (80 mL, 4:1) and was transferred to a reparatory funnel containing saturated aqueous NaHCO₃ solution (50 mL). The aqueous layer was extracted with ethyl acetate/ethanol (4:1) (3×80 mL). The combined organic layers were washed with brine (50 mL), dried over MgSO₄, filtered and concentrated. The residue was suspended in CH₂Cl₂ (25 mL) and the solid (unreacted starting material) was removed by filtration and collected on a Buchner funnel. The filtrate was concentrated and was loaded onto a column with CH₂Cl₂ with a small amount of methanol. The residue was purified by column chromatography on silica gel (50%→70% ethyl acetate containing 1% methanol in hexanes) to afford ethyl 6-chloroimidazo[1,2-b]pyridazine-3-carboxylate (1.73 g, 58% yield) as a colorless solid: ¹H NMR (400 MHz, CDCl₃) δ 8.34 (s, 1H), 7.99 (d, J=9.6 Hz, 1H), 7.24 (d, J=9.6 Hz, 1H), 4.44 (q, J=7.2 Hz, 2H), 1.41 (t, J=7.2 Hz, 3H); LCMS (ESI) m/e 226.2 [(M+H)⁺, calcd for C₉H₉N₃O₂Cl 226.0].

### Part C. 6-Chloroimidazo[1,2-b]pyridazine-3-carboxylic acid

A solution of ethyl 6-chloroimidazo[1,2-b]pyridazine-3-carboxylate (1.79 g, 7.93 mmol) in HCl (6 N) (25.0 mL, 823 mmol) in pressure vessel was heated at 90° C. for 15 h. A white precipitate formed. The mixture was cooled to 0° C. and the solid was collected on a Buchner funnel and was washed with water. The solid was dried under vacuum to give 6-chloroimidazo[1,2-b]pyridazine-3-carboxylic acid (1.5 g, 96% yield) as a colorless solid: ¹H NMR (400 MHz, DMSO-d₆) δ 13.25 (br s, 1H), 8.37 (s, 1H), 8.37 (d, J=9.6 Hz, 1H), 7.61 (d, J=9.6 Hz, 1H); LCMS (ESI) m/e 198.1 [(M+H)⁺, calcd for C₇H₅N₃O₂Cl 198.0].

### Part D. 6-Chloro-N-(3-(5-isopropyl-1,2,4-oxadiazol-3-yl)phenyl)imidazo[1,2-b]pyridazine-3-carboxamide

To a mixture of 6-chloroimidazo[1,2-b]pyridazine-3-carboxylic acid (1.22 g, 6.17 mmol) and 3-(5-isopropyl-1,2,4-oxadiazol-3-yl)aniline (1.882 g, 9.26 mmol) in CH₂Cl₂ (30 mL) at room temperature was added N,N-diisopropylethylamine (5.4 mL, 31 mmol) and HATU (3.52 g, 9.26 mmol). The reaction mixture was stirred at room temperature for 12 hours. The mixture was transferred to a separatory funnel containing saturated aqueous NaHCO₃ solution (50 mL) and was extracted with CH₂Cl₂ (3×50 mL). The organic layer was concentrated and the residue was purified by column chromatography on silica gel (30%→40% ethyl acetate in hexanes) to afford 6-chloro-N-(3-(5-isopropyl-1,2,4-oxadiazol-3-yl)phenyl)imidazo[1,2-b]pyridazine-3-carboxamide (1.4 g, 59% yield) as a yellow solid: ¹H NMR (400 MHz, CD₃OD) δ 8.40-8.45 (m, 2H), 8.23 (d, J=9.6 Hz, 1H), 7.78-7.86 (m, 2H), 7.55 (d, J=9.3 Hz, 1H), 7.50 (t, J=7.9 Hz, 1H), 3.33 (q, J=7.1 Hz, 1H), 1.45 (d, J=7.1 Hz, 6H); LRMS (ESI) m/e 383.3 [(M+H)⁺, calcd for C₁₈H₁₆N₆O₂Cl 383.1].

### Part E. 6-(trans-4-Aminocyclohexylamino)-N-(3-(5-isopropyl-1,2,4-oxadiazol-3-yl)phenyl)imidazo[1,2-b]pyridazine-3-carboxamide

A mixture of 6-chloro-N-(3-(5-isopropyl-1,2,4-oxadiazol-3-yl)phenyl)imidazo[1,2-b]pyridazine-3-carboxamide (220 mg, 0.575 mmol) and cyclohexane-1,4-diamine (459 mg, 4.02 mmol) in a pressure vessel was heated to 150° C. for 2.5 h. The material was transferred to a separatory funnel containing water. The aqueous layer was extracted with CH₂Cl₂ (3×20 mL). The combined organic layers were concentrated and the residue was purified by column chromatography on silica gel (20% methanol with NH₃ (2M) in CH₂Cl₂) to afford 6-(trans-4-aminocyclohexylamino)-N-(3-(5-isopropyl-1,2,4-oxadiazol-3-yl)phenyl)imidazo[1,2-b]pyridazine-3-carboxamide (180 mg, 67% yield) as a yellow solid: ¹H NMR (400 MHz, DMSO-d₆) δ 10.92 (s, 1H), 8.40 (s, 1H), 8.05 (s, 1H), 7.90-7.87 (m, 2H), 7.80 (d, J=7.2 Hz, 1H), 7.61 (t, J=8.0 Hz, 1H), 7.48 (d, J=7.2 Hz, 1H), 6.87 (d, J=9.6 Hz, 1H), 3.73-3.68 (m, 1H), 3.38 (q, J=7.2 Hz, 1H), 3.33 (br s, 2H), 2.58-2.53 (m, 1H), 2.15 (d, J=10.8 Hz, 2H), 1.79 (d, J=11.6 Hz, 2H), 1.40 (d, J=6.8 Hz, 6H), 1.35-1.26 (m, 2H), 1.23-1.15 (m, 2H); LRMS (ESI) m/e 461.2 [(M+H)⁺, calcd for C₂₄H₂₉N₈O₂ 461.2]. HPLC retention time (method A): t_{R}=8.11 min; HPLC retention time (method B): t_{R}=8.27 min.

### Example 12

### 6-(trans-4-Aminocyclohexylamino)-N-(3-(5-isopropyl-1,3,4-thiadiazol-2-yl)phenyl)imidazo[1,2-b]pyridazine-3-carboxamide

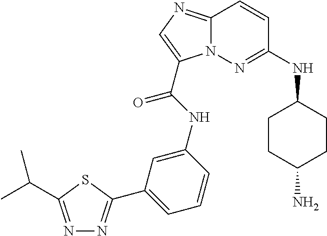 [SMILES: CC(C)c1nnc(-c2cccc(NC(=O)c3cnc4ccc(N[C@H]5CC[C@H](N)CC5)nn34)c2)s1]

Prepared by the method described in Example 11 using 3-(5-isopropyl-1,3,4-thiadiazol-2-yl)aniline in Part D followed by purification by HPLC (acetonitrile with 0.1% TFA/water with 0.1% TFA) to give 6-(trans-4-aminocyclohexylamino)-N-(3-(5-isopropyl-1,3,4-thiadiazol-2-yl)phenyl)imidazo[1,2-b]pyridazine-3-carboxamide (48 mg) as a TFA salt: ¹H NMR (400 MHz, DMSO-d₆) δ 10.81 (s, 1H), 8.56 (s, 1H), 8.09 (s, 1H), 7.94 (s, 1H), 7.91 (m, 3H), 7.68-7.75 (m, 2H), 7.59-7.68 (m, 1H), 7.55 (d, J=7.3 Hz, 1H), 6.91 (d, J=9.8 Hz, 1H), 3.77 (br s, 1H), 3.52 (dt, J=13.8, 6.8 Hz, 1H), 3.14 (br s, 1H) 2.24 (d, J=11.1 Hz, 2H), 2.02 (d, J=11.1 Hz, 2H), 1.51-1.64 (m, 2H), 1.44 (s, 6H), 1.33-1.41 (m, 2H); LRMS (ESI) m/e 477.2 [(M+H)⁺, calcd for C₂₄H₂₉N₈OS 477.2]. HPLC retention time (method A): t_{R}=8.30 min; HPLC retention time (method B): t_{R}=8.35 min.

### Example 13

### 6-(trans-4-Aminocyclohexylamino)-N-(3-(2-isopropyl-2H-tetrazol-5-yl)phenyl)imidazo[1,2-b]pyridazine-3-carboxamide

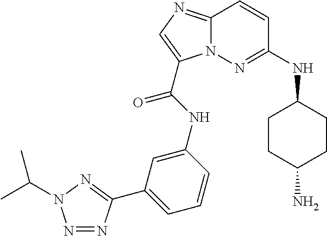 [SMILES: CC(C)n1nnc(-c2cccc(NC(=O)c3cnc4ccc(N[C@H]5CC[C@H](N)CC5)nn34)c2)n1]

Prepared by the method described in Example 11 using 3-(2-isopropyl-2H-tetrazol-5-yl)aniline in Part D to give 6-(trans-4-aminocyclohexylamino)-N-(3-(2-isopropyl-2H-tetrazol-5-yl)phenyl)imidazo[1,2-b]pyridazine-3-carboxamide (55 mg) as a yellow solid: ¹H NMR (400 MHz, CD₃OD) δ 8.19 (br s, 1H), 7.94 (s, 1H), 7.90 (d, J=7.6 Hz, 1H), 7.68 (d, J=7.3 Hz, 1H), 7.55 (d, J=9.6 Hz, 1H), 7.38 (t, J=7.8 Hz, 1H), 6.69 (d, J=9.6 Hz, 1H), 5.12 (ddd, J=13.1, 6.4, 6.2 Hz, 1H), 3.66 (t, J=10.2 Hz, 1H), 2.61 (t, J=10.7 Hz, 1H), 2.23 (d, J=10.1 Hz, 2H), 1.89 (d, J=10.3 Hz, 2H), 1.68 (d, J=6.3 Hz, 6H), 1.42-1.57 (m, 2H), 1.26-1.39 (m, 2H); LRMS (ESI) m/e 461.3 [(M+H)⁺, calcd for C₂₃H₂₉N₁₀O 461.3]. HPLC retention time (method A): t_{R}=7.79 min; HPLC retention time (method B): t_{R}=7.81 min.

### Example 14

### 6-(trans-4-Aminocyclohexylamino)-N-(3-(2-isopropyloxazol-4-yl)phenyl)imidazo[1,2-b]pyridazine-3-carboxamide

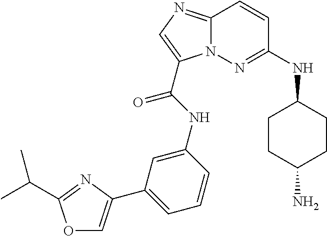 [SMILES: CC(C)c1nc(-c2cccc(NC(=O)c3cnc4ccc(N[C@H]5CC[C@H](N)CC5)nn34)c2)co1]

Prepared by the method described in Example 11 using 3-(2-isopropyloxazol-4-yl)aniline in Part D followed by purification by HPLC (acetonitrile with 0.1% TFA/water with 0.1% TFA) to give 6-(trans-4-aminocyclohexylamino)-N-(3-(2-isopropyloxazol-4-yl)phenyl)imidazo[1,2-b]pyridazine-3-carboxamide (26 mg) as a TFA salt: ¹H NMR (400 MHz, DMSO-d₆) δ 10.70 (s, 1H), 8.50 (s, 1H), 8.44 (s, 1H), 8.08 (s, 1H), 7.93 (d, J=9.6 Hz, 1H), 7.81 (d, J=3.5 Hz, 3H), 7.55-7.59 (m, 1H), 7.48-7.55 (m, 2H), 7.26 (d, J=8.1 Hz, 1H), 6.91 (d, J=9.8 Hz, 1H), 3.70 (br s, 1H), 3.15 (quin, J=7.1 Hz, 1H), 3.10 (br s, 1H), 2.24 (d, J=9.3 Hz, 2H), 2.00 (d, J=9.6 Hz, 2H), 1.35-1.48 (m, 4H), 1.33 (d, J=6.8 Hz, 6H); LRMS (ESI) m/e 460.3 [(M+H)⁺, calcd for C₂₅H₃₀N₇O₂ 460.3]. HPLC retention time (method A): t_{R}=7.87 min; HPLC retention time (method B): t_{R}=8.13 min.

### Example 15

### 6-(trans-4-Aminocyclohexylamino)-N-(3-(2-methyloxazol-4-yl)phenyl)imidazo[1,2-b]pyridazine-3-carboxamide

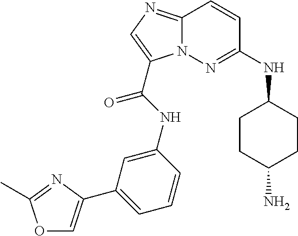 [SMILES: Cc1nc(-c2cccc(NC(=O)c3cnc4ccc(N[C@H]5CC[C@H](N)CC5)nn34)c2)co1]

Prepared by the method described in Example 11 using 3-(2-methyloxazol-4-yl)aniline in Part D to give 6-(trans-4-aminocyclohexylamino)-N-(3-(2-methyloxazol-4-yl)phenyl)imidazo[1,2-b]pyridazine-3-carboxamide (60 mg) as a yellow solid: ¹H NMR (400 MHz, DMSO-d₆) δ 10.85 (s, 1H), 8.51 (s, 1H), 8.07 (s, 1H), 8.04 (s, 1H), 7.89 (d, J=9.8 Hz, 1H), 7.71 (d, J=8.1 Hz, 1H), 7.42-7.57 (m, 3H), 6.87 (d, J=9.8 Hz, 1H), 3.67-3.78 (m, 1H), 2.52-2.59 (m, 1H), 2.49 (s, 3H), 2.15 (d, J=11.6 Hz, 2H), 1.77 (d, J=11.3 Hz, 2H), 1.14-1.36 (m, 4H); LRMS (ESI) m/e 432.4 [(M+H)⁺, calcd for C₂₃H₂₆N₇O₂ 432.2]. HPLC retention time (method A): t_{R}=7.07 min; HPLC retention time (method B): t_{R}=7.10 min.

### Example 16

### 6-(trans-4-Aminocyclohexylamino)-N-(3-(2-phenyloxazol-4-yl)phenyl)imidazo[1,2-b]pyridazine-3-carboxamide

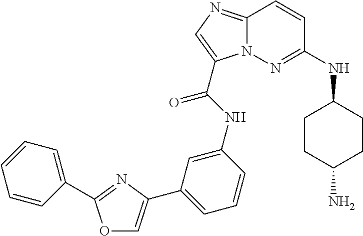 [SMILES: N[C@H]1CC[C@H](Nc2ccc3ncc(C(=O)Nc4cccc(-c5coc(-c6ccccc6)n5)c4)n3n2)CC1]

Prepared by the method described in Example 11 using 3-(2-phenyloxazol-4-yl)aniline in Part D to give 6-(trans-4-aminocyclohexylamino)-N-(3-(2-phenyloxazol-4-yl)phenyl)imidazo[1,2-b]pyridazine-3-carboxamide (87 mg) as a yellow solid: ¹H NMR (400 MHz, DMSO-d₆) δ 10.90 (s, 1H), 8.78 (s, 1H), 8.29 (s, 1H), 8.06-8.10 (m, 2H), 8.06 (s, 1H), 7.91 (d, J=9.8 Hz, 1H), 7.48-7.68 (m, 7H), 6.88 (d, J=9.8 Hz, 1H), 3.69-3.78 (m, 1H), 2.52-2.57 (m, 1H), 2.15 (d, J=10.6 Hz, 2H), 1.74 (d, J=10.3 Hz, 2H), 1.23-1.35 (m, 2H), 1.07-1.20 (m, 2H); LRMS (ESI) m/e 494.3 [(M+H)⁺, calcd for C₂₈H₂₈N₇O₂ 494.2]. HPLC retention time (method A): t_{R}=8.60 min; HPLC retention time (method B): t_{R}=8.91 min.

### Example 17

### N⁶-(trans-4-Aminocyclohexyl)-N⁸-phenyl-3-(thiophen-3-yl)imidazo[1,2-b]pyridazine-6,8-diamine

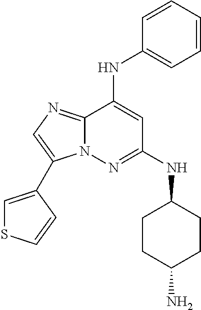 [SMILES: N[C@H]1CC[C@H](Nc2cc(Nc3ccccc3)c3ncc(-c4ccsc4)n3n2)CC1]

### Part A. 3,8-Dibromo-6-chloroimidazo[1,2-b]pyridazine

A solution of 8-bromo-6-chloroimidazo[1,2-b]pyridazine (5.00 g, 21.51 mmol) (prepared as described by Vaccaro W. et al. United States Patent Appl. US 2008/0045536 A1, 2008) and NBS (4.21 g, 23.7 mmol) in CH₂Cl₂ (40 mL) was heated to 70° C. in a sealed tube. The solvent was evaporated and water was added to the residue resulting in a brown ppt which was filtered and dried to afford 3,8-dibromo-6-chloroimidazo[1,2-b]pyridazine (6.7 g, 100% yield) as a brown solid: ¹H NMR (400 MHz, CDCl₃) δ 7.82 (s, 1H), 7.42 (s, 1H); LRMS (ESI) m/e 313.8 [(M+H)⁺, calcd for C₆H₃N₃Br₂Cl 312.4].

### Part B. 3-Bromo-6-chloro-8-ethoxyimidazo[1,2-b]pyridazine

To a solution of 3,8-dibromo-6-chloroimidazo[1,2-b]pyridazine (9.00 g, 28.9 mmol) in ethanol (50 mL) at 0° C. was added sodium ethanolate (21.6 mL, 57.8 mmol) dropwise. After 1 h, the reaction was complete. The solvent was evaporated. The residue was treated with 1 N NH₄Cl in water and extracted with CH₂Cl₂ (3×30 mL). The combined organic layers were concentrated and the residue was purified by column chromatography on silica gel (30%→40% ethyl acetate in hexanes) to afford 3-bromo-6-chloro-8-ethoxyimidazo[1,2-b]pyridazine (6.00 g, 75% yield) as a yellow solid: ¹H NMR (400 MHz, CDCl₃) δ 7.62 (s, 1H), 6.43 (s, 1H), 4.35 (q, J=7.1 Hz, 2H), 1.58 (t, J=7.1 Hz, 3H); LRMS (ESI) m/e 278.0 [(M+H)⁺, calcd for C₈H₈N₃BrClO 277.5].

### Part C. 6-Chloro-8-ethoxy-3-(thiophen-3-yl)imidazo[1,2-b]pyridazine

N₂ gas was bubbled to a mixture of 3-bromo-6-chloro-8-ethoxyimidazo[1,2-b]pyridazine (1.55 g, 5.61 mmol), 3-thiophene boronic acid (0.789 g, 6.17 mmol), Na₂CO₃ (2 M) (4.20 mL, 8.41 mmol), toluene (24 mL), and MeOH (4.80 mL) for 2 min. To this mixture, Pd(PPh₃)₄ (0.972 g, 0.841 mmol) was added and the reaction was heated to 90° C. for 20 h. The reaction was cooled to room temperature and was transferred to reparatory funnel containing brine (50 mL) and the aqueous layer was extracted with ethyl acetate (3×50 mL). The combined organic layers were concentrated and the residue was purified by column chromatography on silica gel (10%→50% ethyl acetate in hexanes) to afford 6-chloro-8-ethoxy-3-(thiophen-3-yl)imidazo[1,2-b]pyridazine (1.3 g, 83% yield) as a yellow solid: LRMS (ESI) m/e 280.0 [(M+H)⁺, calcd for C₁₂H₁₁N₃OSCl 280.0].

### Part D. 6-Chloro-N-phenyl-3-(thiophen-3-yl)imidazo[1,2-b]pyridazin-8-amine

To a solution of 6-chloro-8-ethoxy-3-(thiophen-3-yl)imidazo[1,2-b]pyridazine (1.25 g, 4.47 mmol) and aniline (0.408 mL, 4.47 mmol) at 0° C. was added LiHMDS (9.4 mL, 9.4 mmol, 1 M in THF) dropwise. The reaction turned dark brown. The solution was allowed to warm to room temperature and stirred for 3 h. The reaction was quenched with brine and the aqueous layer was extracted with ethyl acetate (3×25 mL). The combined organic layers were concentrated and the residue was purified by column chromatography on silica gel (30→40% ethyl acetate in hexanes) to afford 6-chloro-N-phenyl-3-(thiophen-3-yl)imidazo[1,2-b]pyridazin-8-amine (1.10 g, 75% yield) as a yellow solid: ¹H NMR (400 MHz, DMSO-d₆) δ 9.99 (br s, 1H), 8.31 (d, J=2.0 Hz, 1H), 8.13 (s, 1H) 7.80 (dd, J=5.0, 0.8 Hz, 1H), 7.73 (dd, J=5.0, 3.0 Hz, 1H), 7.47 (d, J=4.3 Hz, 4H), 7.25 (ddd, J=8.3, 4.5, 4.3 Hz, 1H), 6.47 (s, 1H); LRMS (ESI) m/e 327.1 [(M+H)⁺, calcd for C₁₂H₁₁N₃OSCl 280.0].

### Part E. N⁶-(trans-4-Aminocyclohexyl)-N⁸-phenyl-3-(thiophen-3-yl)imidazo[1,2-b]pyridazine-6,8-diamine

6-Chloro-N-phenyl-3-(thiophen-3-yl)imidazo[1,2-b]pyridazin-8-amine (370 mg, 1.13 mmol) and trans-1,4-diaminocyclohexane (1.29 g, 11.3 mmol) were heated to 240° C. in microwave for 4 h. The reaction mixture was diluted with water and was transferred to a separatory funnel and was extracted with ethyl acetate (3×30 mL). The combined organic layers were concentrated and the residue was purified by column chromatography on silica gel (5%→20% MeOH with NH₃ (2 M) in CH₂Cl₂) to afford a colorless solid.

In the case of Example 17, the solid was dissolved in 10 mL methylene chloride and was treated with 2 M HCl in ether (5 mL). The white ppt was collected by filtration and dried in vacuo to afford N⁶-(trans-4-aminocyclohexyl)-N⁸-phenyl-3-(thiophen-3-yl)imidazo[1,2-b]pyridazine-6,8-diamine (230 mg, 51% yield) as the HCl salt: ¹H NMR (400 MHz, DMSO-d₆) δ 10.14 (s, 1H), 8.60 (s, 1H), 8.46 (s, 1H), 8.21 (br s, 3H), 7.80 (s, 2H), 7.48 (t, J=7.7 Hz, 2H), 7.39-7.44 (m, 2H), 7.23 (t, J=7.2 Hz, 1H), 6.65 (s, 1H), 3.60 (t, J=11.1 Hz, 1H), 3.06 (br s, 1H), 2.16 (d, J=10.8 Hz, 2H), 2.06 (d, J=10.8 Hz, 2H), 1.46-1.61 (m, 2H), 1.22-1.35 (m, 2H); LRMS (ESI) m/e 405.3 [(M+H)⁺, calcd for C₂₂H₂₅N₆S 405.2]. HPLC retention time (method A): t_{R}=8.12 min; HPLC retention time (method B): t_{R}=8.31 min.

### Example 18

### N⁶-(trans-4-Aminocyclohexyl)-N⁸-mesityl-3-(thiophen-3-yl)imidazo[1,2-b]pyridazine-6,8-diamine

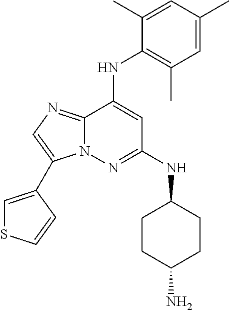 [SMILES: Cc1cc(C)c(Nc2cc(N[C@H]3CC[C@H](N)CC3)nn3c(-c4ccsc4)cnc23)c(C)c1]

Prepared by the method described in Example 17 using 2,4,6-trimethylaniline in Part D to give N⁶-(trans-4-aminocyclohexyl)-N⁸-mesityl-3-(thiophen-3-yl)imidazo[1,2-b]pyridazine-6,8-diamine (20 mg) as a green solid: ¹H NMR (500 MHz, CD₃OD) δ 8.39 (dd, J=12.5, 1.8 Hz, 1H), 7.71 (br s, 1H), 7.70 (s, 1H), 7.48-7.52 (m, 1H), 7.02 (s, 2H), 5.11 (d, J=4.0 Hz, 1H), 3.63-3.76 (m, 1H), 3.36-3.44 (m, 1H), 2.32 (s, 3H), 2.229 (s, 3H), 2.226 (s, 3H), 1.98 (d, J=11.6 Hz, 2H), 1.74 (d, J=11.9 Hz, 2H), 1.56-1.67 (m, 2H), 1.32-1.43 (m, 2H); LRMS (ESI) m/e 447.3 [(M+H)⁺, calcd for C₂₅H₃₁N₆S 447.3]. HPLC retention time (method A): t_{R}=8.85 min; HPLC retention time (method B): t_{R}=9.21 min.

### Example 19

### N⁶-(trans-4-(Dimethylamino)cyclohexyl)-N⁸-phenyl-3-(thiophen-3-yl)imidazo[1,2-b]pyridazine-6,8-diamine

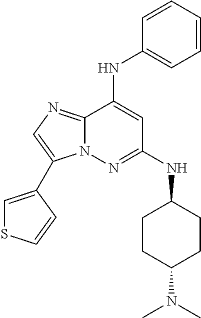 [SMILES: CN(C)[C@H]1CC[C@H](Nc2cc(Nc3ccccc3)c3ncc(-c4ccsc4)n3n2)CC1]

Prepared by the method described in Example 17 using trans-N¹,N¹-dimethylcyclohexane-1,4-diamine in Part E followed by purification by HPLC (acetonitrile with 0.1% TFA/water with 0.1% TFA) to give N⁶-(trans-4-(dimethylamino)cyclohexyl)-N⁸-phenyl-3-(thiophen-3-yl)imidazo[1,2-b]pyridazine-6,8-diamine (5 mg) as a TFA salt: ¹H NMR (400 MHz, DMSO-d₆) δ 9.49 (br s, 1H), 9.05 (s, 1H), 8.39 (dd, J=3.0, 1.0 Hz, 1H), 8.08 (s, 1H), 7.78 (dd, J=5.0, 1.0 Hz, 1H), 7.71 (dd, J=5.3, 3.0 Hz, 1H), 7.46 (t, J=7.8 Hz, 2H), 7.39-7.43 (m, 2H), 7.16-7.21 (m, 1H), 6.77 (br s, 1H), 6.44 (s, 1H), 4.06 (br s, 1H), 3.21 (br s, 1H), 2.74 (s, 3H), 2.73 (s, 3H), 2.11 (d, J=9.6 Hz, 2H), 1.82 (d, J=7.8 Hz, 2H), 1.65-1.74 (m, 4H); LRMS (ESI) m/e 433.3 [(M+H)⁺, calcd for C₂₄H₂₉N₆S 433.2]. HPLC retention time (method A): t_{R}=8.53 min; HPLC retention time (method B): t_{R}=9.10 min.

### Example 20

### N⁶-(trans-4-Aminocyclohexyl)-N⁸-methyl-3-(thiophen-3-yl)imidazo[1,2-b]pyridazine-6,8-diamine

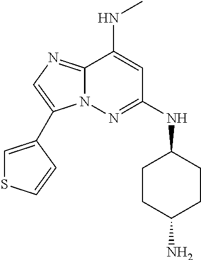 [SMILES: CNc1cc(N[C@H]2CC[C@H](N)CC2)nn2c(-c3ccsc3)cnc12]

Prepared by the method described in Example 17 using methanamine in Part D to give N⁶-(trans-4-aminocyclohexyl)-N⁸-methyl-3-(thiophen-3-yl)imidazo[1,2-b]pyridazine-6,8-diamine (5 mg) as a colorless solid: ¹H NMR (400 MHz, CDCl₃) δ 8.24 (dd, J=3.0, 1.3 Hz, 1H), 7.57 (dd, J=5.2, 1.1 Hz, 1H), 7.54 (s, 1H), 7.36 (dd, J=5.0, 3.0 Hz, 1H), 5.56 (d, J=4.8 Hz, 1H), 5.30 (s, 1H), 3.94 (d, J=7.6 Hz, 1H), 3.68-3.77 (m, 1H), 2.95 (d, J=5.0 Hz, 3H), 2.71-2.78 (m, 1H), 2.27 (d, J=12.1 Hz, 2H), 1.95 (d, J=11.8 Hz, 2H), 1.15-1.36 (m, 4H); LRMS (ESI) m/e 343.3 [(M+H)⁺, calcd for C₁₇H₂₃N₆S 343.2]. HPLC retention time (method A): t_{R}=6.01 min; HPLC retention time (method B): t_{R}=4.82 min.

### Example 21

### N⁶-(trans-4-Aminocyclohexyl)-N⁸-cyclopropyl-3-(thiophen-3-yl)imidazo[1,2-b]pyridazine-6,8-diamine

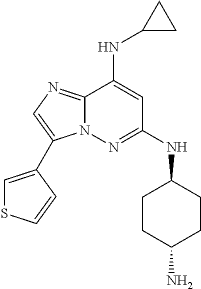 [SMILES: N[C@H]1CC[C@H](Nc2cc(NC3CC3)c3ncc(-c4ccsc4)n3n2)CC1]

Prepared by the method described in Example 17 using cyclopropanamine in Part D to give N⁶-(trans-4-aminocyclohexyl)-N⁸-cyclopropyl-3-(thiophen-3-yl)imidazo[1,2-b]pyridazine-6,8-diamine (26 mg) as a yellow semi-solid: ¹H NMR (400 MHz, CDCl₃) δ 8.23 (dd, J=3.0, 1.3 Hz, 1H), 7.56 (dd, J=5.0, 1.3 Hz, 1H), 7.52 (s, 1H), 7.35 (dd, J=5.0, 3.0 Hz, 1H), 5.82 (s, 1H), 5.68 (s, 1H), 4.05 (d, J=7.3 Hz, 1H), 3.67-3.77 (m, 1H), 2.69-2.78 (m, 1H), 2.47-2.55 (m, 1H), 2.27 (d, J=11.6 Hz, 2H), 1.94 (d, J=11.8 Hz, 2H), 1.18-1.38 (m, 4H), 0.74-0.81 (m, 2H), 0.58-0.65 (m, 2H); LRMS (ESI) m/e 369.4 [(M+H)⁺, calcd for C₁₉H₂₅N₆S 369.2]. HPLC retention time (method A): t_{R}=7.33 min; HPLC retention time (method B): t_{R}=7.65 min.

### Example 22

### N⁶-(trans-4-Aminocyclohexyl)-N⁸-(4-methoxyphenyl)-3-(thiophen-3-yl)imidazo[1,2-b]pyridazine-6,8-diamine

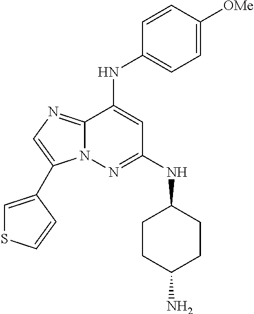 [SMILES: COc1ccc(Nc2cc(N[C@H]3CC[C@H](N)CC3)nn3c(-c4ccsc4)cnc23)cc1]

Prepared by the method described in Example 17 using 4-methoxyaniline in Part D to give N⁶-(trans-4-aminocyclohexyl)-N⁸-(4-methoxyphenyl)-3-(thiophen-3-yl)imidazo[1,2-b]pyridazine-6,8-diamine (6 mg) as a colorless solid: ¹H NMR (400 MHz, CDCl₃) δ 8.25 (d, J=1.8 Hz, 1H), 7.56-7.66 (m, 2H), 7.38 (dd, J=4.5, 3.0 Hz, 1H), 7.16-7.23 (m, 3H), 6.92 (d, J=8.8 Hz, 2H), 5.68 (s, 1H), 3.88 (d, J=7.1 Hz, 1H), 3.81 (s, 3H), 3.70 (br s, 1H), 2.72 (t, J=10.6 Hz, 1H), 2.24 (d, J=11.6 Hz, 2H), 1.92 (d, J=11.1 Hz, 2H), 1.13-1.38 (m, 4H); LRMS (ESI) m/e 435.4 [(M+H)⁺, calcd for C₂₃H₂₇N₆OS 435.2]. HPLC retention time (method A): t_{R}=8.13 min; HPLC retention time (method B): t_{R}=8.43 min.

### Example 23

### N⁶-(trans-4-Aminocyclohexyl)-N⁸-(4-(5-isopropyl-1,3,4-thiadiazol-2-yl)phenyl)-3-(thiophen-3-yl)imidazo[1,2-b]pyridazine-6,8-diamine

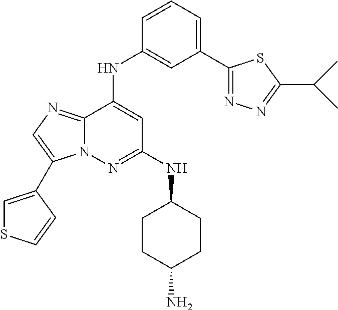 [SMILES: CC(C)c1nnc(-c2cccc(Nc3cc(N[C@H]4CC[C@H](N)CC4)nn4c(-c5ccsc5)cnc34)c2)s1]

Prepared by the method described in Example 17 using 3-(5-isopropyl-1,3,4-thiadiazol-2-yl)aniline in Part D to give N⁶-(trans-4-aminocyclohexyl)-N⁸-(4-(5-isopropyl-1,3,4-thiadiazol-2-yl)phenyl)-3-(thiophen-3-yl)imidazo[1,2-b]pyridazine-6,8-diamine (15 mg) as a colorless solid: ¹H NMR (400 MHz, CDCl₃) δ 8.26 (d, J=1.5 Hz, 1H), 8.03 (br s, 1H), 7.62 (br s, 1H), 7.60 (d, J=5.0 Hz, 1H), 7.54 (d, J=7.3 Hz, 1H), 7.43 (t, J=7.8 Hz, 1H), 7.36-7.41 (m, 1H), 7.31 (d, J=7.6 Hz, 1H), 7.24 (s, 1H), 6.14 (s, 1H), 4.25 (d, J=6.8 Hz, 1H), 3.73 (br s, 1H), 3.49 (dt, J=13.8, 6.9 Hz, 1H), 2.74 (br s, 1H), 2.27 (d, J=10.8 Hz, 2H), 1.94 (d, J=11.3 Hz, 2H), 1.47 (d, J=6.8 Hz, 6H), 1.15-1.37 (m, 4H); LRMS (ESI) m/e 531.4 [(M+H)⁺, calcd for C₂₇H₃₁N₈S₂ 531.2]. HPLC retention time (method A): t R=9.04 min; HPLC retention time (method B): t_{R}=9.18 min.

### BIOLOGICAL DATA

### Methods

### AAK1 Kinase Assay

The assays were performed in U-bottom 384-well plates. The final assay volume was 30 μl prepared from 15 μl additions of enzyme and substrates (fluoresceinated peptide (5-FAM)-Aha-KEEQSQITSQVTGQIGWR-NH2 and ATP) and test compounds in assay buffer (10 mM Tris-HCL pH 7.4, 10 mM MgCl₂, 0.01% Tween-20 and 1.0 mM DTT). The reactions were initiated by the combination of bacterially expressed, GST-Xa-hAAK1 with substrates and test compounds. The reactions were incubated at room temperature for 3 hours and terminated by adding 60 μl of 35 mM EDTA buffer to each sample. The reactions were analyzed on the Caliper LabChip 3000 (Caliper, Hopkinton, Mass.) by electrophoretic separation of the fluorescent substrate and phosphorylated product. Inhibition data were calculated by comparison to EDTA quenched control reactions for 100% inhibition and vehicle-only reactions for 0% inhibition. The final concentration of reagents in the assays are ATP, 22 μM; (5-FAM)-Aha-KEEQSQITSQVTGQIGWR-NH2, 1.5 μM; GST-Xa-hAAK1, 3.5 nM; and DMSO, 1.6%. Dose response curves were generated to determine the concentration required inhibiting 50% of kinase activity (IC₅₀). Compounds were dissolved at 10 mM in dimethylsulfoxide (DMSO) and evaluated at eleven concentrations. IC₅₀ values were derived by non-linear regression analysis. Results are shown in Table 3.

TABLE 3

| Example | IC₅₀ |
|---|---|
| 1 | 10 |
| 2 | 11 |
| 3 | 94 |
| 4 | 5.1 |
| 5 | 79 |
| 6 | 940 |
| 7 | 114 |
| 8 | 8.9 |
| 9 | 8.0 |
| 10 | 16 |
| 11 | 27 |
| 12 | 9.1 |
| 13 | 12 |
| 14 | 158 |
| 15 | 342 |
| 16 | 154 |
| 17 | 10.0 |
| 18 | |
| 19 | 12 |
| 20 | 38 |
| 21 | 40 |
| 22 | 9.0 |
| 23 | 39 |

### AAK1 Knockout Mice

Mice homozygous (−/−) for the disruption of the AAK1 gene were prepared by two methods; gene trapping and homologous recombination.

Gene trapping is a method of random insertional mutagenesis that uses a fragment of DNA coding for a reporter or selectable marker gene as a mutagen. Gene trap vectors have been designed to integrate into introns or genes in a manner that allows the cellular splicing machinery to splice vector encoded exons to cellular mRNAs. Commonly, gene trap vectors contain selectable marker sequences that are preceded by strong splice acceptor sequences and are not preceded by a promoter. Thus, when such vectors integrate into a gene, the cellular splicing machinery splices exons from the trapped gene onto the 5′ end of the selectable marker sequence. Typically, such selectable marker genes can only be expressed if the vector encoding the gene has integrated into an intron. The resulting gene trap events are subsequently identified by selecting for cells that can survive selective culture.

Embryonic stem cells (Lex-1 cells from derived murine strain A129), were mutated by a process involving the insertion of at least a portion of a genetically engineered vector sequence into the gene of interest, the mutated embryonic stem cells were microinjected into blastocysts which were subsequently introduced into pseudopregnant female hosts and carried to term using established methods. See, e.g., “Mouse Mutagenesis”, 1998, Zambrowicz et al., eds., Lexicon Press, The Woodlands, Tex. The resulting chimeric animals were subsequently bred to produce offspring capable of germline transmission of an allele containing the engineered mutation in the gene of interest.

AAK1-gene disrupted mice were also made by homologous recombination. In this case, the second coding exon of the murine AAK1 gene (see GenBank Accession Number NM 177762) was removed by methods known in the art. See, e.g., U.S. Pat. Nos. 5,487,992, 5,627,059, and 5,789,215.

Mice homozygous (−/−) for the disruption of the AAK1 gene were studied in conjunction with mice heterozygous (+/−) for the disruption of the AAK1 gene, and wild-type (+/+) litter mates. During this analysis, the mice were subject to a medical work-up using an integrated suite of medical diagnostic procedures designed to assess the function of the major organ systems in a mammalian subject. Homozygous (−/−) “knockout” mice were studied in conjunction with their heterozygous (+/−) and wild-type (+/+) litter mates. Disruption of the AAK1 gene was confirmed by Southern analysis. Expression of the murine homolog of AAK1 was detected by RT-PCR in murine brain; spinal cord; eye; thymus; spleen; lung; kidney; liver; skeletal muscle; bone; stomach, small intestine and colon; heart; adipose; asthmatic lung; LPS liver; blood; banded heart; aortic tree; prostate; and mammary gland (5 week virgin, mature virgin, 12 DPC, 3 day post-partum (lactating), 3 day post-weaning (early involution), and 7 day post-weaning (late involution)).

AAK1 homozygous (−/−) and their wild-type (+/+) littermates were tested using the formalin paw test in order to assess their acute and tonic nociceptive responses. For these tests, Automatic Nociception Analyzers (purchased from the Ozaki lab at University of California, San Diego) were used. A metal band was placed around the left hind paw of each mouse 30 minutes prior to testing. After the 30-minute acclimation period, 20 μl of 5% formalin is subcutaneously injected in the dorsal surface of the left hind paw. Mice were individually housed in cylindrical chambers for 45 minutes. Fresh 5% formalin solution was prepared by diluting formaldehyde (Formalde-fresh 20%, Fisher Scientific, Fair Lawn, N.J.) with distilled water. Investigatory compounds were administered 30 minutes prior to formalin injection.

A computer recorded flinches per minute, total flinches for phase I (acute phase=first 8 minutes), and total flinches for phase II (tonic phase=time between minutes 20-40 or 10-60 minutes for drug studies) through an electromagnetic field. See Yaksh T L, Ozaki G, McCumber D, Rathbun M, Svensson C, Malkmus S, Yaksh M C. An automated flinch detecting system for use in the formalin nociceptive bioassay. J Appl Physiol., 2001; 90:2386-402. As shown in FIG. 1, phase 1 and phase 2 data were obtained using homozygous (−/−) mice females (n=16), wild-type females (n=15), homozygous (−/−) mice males (n=9), and wild-type males (n=18). In all groups and in both phases, the AAK1 homozygous (−/−) mice exhibited significantly less recorded paw flinching than their wild-type (+/+) littermates.

Studies of AAK1 knockout mice showed that disruption of the AAK1 gene affects pain response as measured using the formalin paw test described above. The same test was used to confirm that the administration of an AAK1 inhibitor can also affect pain response.

A compound of the disclosure was tested in this assay at different doses. Gabapentin and pregabalin were used as positive controls. Results are shown below in Table 4, wherein the effect of gabapentin at 200 mg/kg is considered a 100% response, the % response for the other compounds is relative to the 200 mg/kg dose of gabapentin, “sc” means subcutaneous administration; “po” means oral administration.

TABLE 4

| | Dose | |
|---|---|---|
| Compound | (mg/kg) | Response |
| Gabapentin | 200 sc | 73% |
| Example 1: 6-(((1R,4R)-4-aminocyclohexyl)amino)- | 60 sc | 59% |
| N-(3-(5-isopropyl-1,2,4-oxadiazol-3- | | |
| yl)phenyl)imidazo[1,2-b]pyridazine-3-carboxamide | | |

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

1. A method for treating or managing a disease or a disorder mediated by AAK1 activity, the method comprising administering to a patient in need thereof a therapeutically effective amount of a compound of formula (I) 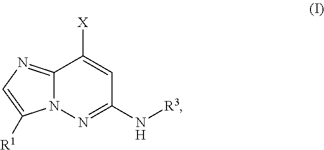
or a pharmaceutically acceptable salt thereof, wherein:
R¹ is selected from —C(O)NHR² and thienyl;
R² is selected from 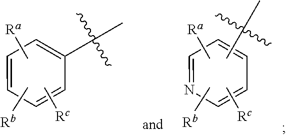
wherein R^{a} and R^{b} are independently selected from hydrogen, C₂-C₄ alkenyl, C₁-C₃alkoxy, C₁-C₃alkoxyC₁-C₃alkyl, C₁-C₃alkyl, cyano, halo, C₁-C₃ haloalkyl, hydroxy, and C₁-C₃hydroxyalkyl; or, alternatively,
when R^{a} and R^{b} are on adjacent carbons, they, together with the carbon atoms to which they are attached, can optionally form a five-membered aromatic ring containing one or two nitrogen atoms;
R^{c} is a five-membered aromatic ring containing one, two, three, or four heteroatoms independently selected from nitrogen, oxygen, and sulfur, wherein the five-membered aromatic ring is optionally substituted with one group selected from C₁-C₄alkoxy, C₁-C₄ alkoxyC₁-C₄alkyl, C₁-C₄alkyl, C₁-C₄ aminoalkyl, cyano, C₃-C₆ cycloalkyl, C₁-C₄ haloalkyl, C₁-C₄ hydroxyalkyl, nitro, and phenyl;
R³ is selected from 4-(C₁-C₃acylamino)cyclohexyl, C₁-C₄-aminoalkyl, 2-aminocyclobutyl, 4-aminocyclohexyl, 3-aminocyclopentyl, 3-aminomethylcyclohexyl, 3-aminomethylcyclopentyl, 2-cyanocyclobutyl, 4-cyanocyclohexyl, cyanomethyl, 2-methylaminocyclobutyl, 4-methylaminocyclohexyl, 3-methylaminocyclopentyl, octahydrocyclopenta[c]pyrrolyl, 4-piperidyl, and 3-azabicyclo[3.2.1]octyl; and
X is selected from hydrogen, C₁-C₃alkylamino, C₃-C₆cycloalkylamino, and phenylamino, wherein the phenylamino is optionally substituted with one group selected from C₁-C₃alkoxy, C₁-C₃alkyl, cyano, and a five-membered aromatic ring containing one, two, or three heteroatoms independently selected from nitrogen, oxygen, and sulfur wherein the five-membered aromatic ring is optionally substituted with one C₁-C₃alkyl group.

2. The method of claim 1, wherein
R² is 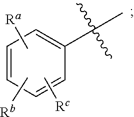
wherein R^{a} and R^{b} are hydrogen;
R^{c} is a five-membered aromatic ring containing one, two, three, or four heteroatoms independently selected from nitrogen, oxygen, and sulfur, wherein the five-membered aromatic ring is optionally substituted with one group selected from C₁-C₄alkoxy, cyano, nitro, and phenyl; and
R³ is 4-aminocyclohexyl.

3. The method of claim 1, wherein the disease or disorder is selected from Alzheimer's disease, bipolar disorder, pain, Parkinson's disease, and schizophrenia.

4. The method of claim 3 wherein the pain is neuropathic pain.

5. The method of claim 4 wherein the neuropathic pain is fibromyalgia or peripheral neuropathy.

6. The method of claim 1 wherein the compound of formula (I) is selected from
6-(trans-4-Aminocyclohexylamino)-N-(3-(5-isopropyl-1,2,4-oxadiazol-3-yl)phenyl)-8-(phenylamino)imidazo[1,2-b]pyridazine-3-carboxamide;
6-(trans-4-Aminocyclohexylamino)-N-(3-(5-isopropyl-1,3,4-thiadiazol-2-yl)phenyl)-8-(phenylamino)imidazo[1,2-b]pyridazine-3-carboxamide;
6-(trans-4-Aminocyclohexylamino)-N-(3-(2-isopropyloxazol-4-yl)phenyl)-8-(phenylamino)imidazo[1,2-b]pyridazine-3-carboxamide;
6-(trans-4-Aminocyclohexylamino)-N-(3-(2-isopropyl-2H-tetrazol-5-yl)phenyl)-8-(phenylamino)imidazo[1,2-b]pyridazine-3-carboxamide;
6-(trans-4-Aminocyclohexylamino)-N-(3-(2-methyloxazol-4-yl)phenyl)-8-(phenylamino)imidazo[1,2-b]pyridazine-3-carboxamide;
6-(trans-4-Aminocyclohexylamino)-8-(phenylamino)-N-(3-(2-phenyloxazol-4-yl)phenyl)imidazo[1,2-b]pyridazine-3-carboxamide;
6-(trans-4-Aminocyclohexylamino)-N-(3-(2-methylthiazol-4-yl)phenyl)-8-(phenylamino)imidazo[1,2-b]pyridazine-3-carboxamide;
6-(trans-4-Aminocyclohexylamino)-N-(3-(5-isopropyl-1,3,4-thiadiazol-2-yl)phenyl)-8-(methylamino)imidazo[1,2-b]pyridazine-3-carboxamide;
6-(trans-4-Aminocyclohexylamino)-N-(3-(2-isopropyl-2H-tetrazol-5-yl)phenyl)-8-(methylamino)imidazo[1,2-b]pyridazine-3-carboxamide;
6-(trans-4-Aminocyclohexylamino)-N-(3-(5-isopropyl-1,2,4-oxadiazol-3-yl)phenyl)-8-(methylamino)imidazo[1,2-b]pyridazine-3-carboxamide;
6-(trans-4-Aminocyclohexylamino)-N-(3-(5-isopropyl-1,2,4-oxadiazol-3-yl)phenyl)imidazo[1,2-b]pyridazine-3-carboxamide;
6-(trans-4-Aminocyclohexylamino)-N-(3-(5-isopropyl-1,3,4-thiadiazol-2-yl)phenyl)imidazo[1,2-b]pyridazine-3-carboxamide;
6-(trans-4-Aminocyclohexylamino)-N-(3-(2-isopropyl-2H-tetrazol-5-yl)phenyl)imidazo[1,2-b]pyridazine-3-carboxamide;
6-(trans-4-Aminocyclohexylamino)-N-(3-(2-isopropyloxazol-4-yl)phenyl)imidazo[1,2-b]pyridazine-3-carboxamide;
6-(trans-4-Aminocyclohexylamino)-N-(3-(2-methyloxazol-4-yl)phenyl)imidazo[1,2-b]pyridazine-3-carboxamide;
6-(trans-4-Aminocyclohexylamino)-N-(3-(2-phenyloxazol-4-yl)phenyl)imidazo[1,2-b]pyridazine-3-carboxamide;
N⁶-(trans-4-Aminocyclohexyl)-N⁸-phenyl-3-(thiophen-3-yl)imidazo[1,2-b]pyridazine-6,8-diamine;
N⁶-(trans-4-Aminocyclohexyl)-N⁸-mesityl-3-(thiophen-3-yl)imidazo[1,2-b]pyridazine-6,8-diamine;
N⁶-(trans-4-(Dimethylamino)cyclohexyl)-N⁸-phenyl-3-(thiophen-3-yl)imidazo[1,2-b]pyridazine-6,8-diamine;
N⁶-(trans-4-Aminocyclohexyl)-N⁸-methyl-3-(thiophen-3-yl)imidazo[1,2-b]pyridazine-6,8-diamine;
N⁶-(trans-4-Aminocyclohexyl)-N⁸-cyclopropyl-3-(thiophen-3-yl)imidazo[1,2-b]pyridazine-6,8-diamine;
N⁶-(trans-4-Aminocyclohexyl)-N⁸-(4-methoxyphenyl)-3-(thiophen-3-yl)imidazo[1,2-b]pyridazine-6,8-diamine; and
N⁶-(trans-4-Aminocyclohexyl)-N⁸-(4-(5-isopropyl-1,3,4-thiadiazol-2-yl)phenyl)-3-(thiophen-3-yl)imidazo[1,2-b]pyridazine-6,8-diamine;
or a pharmaceutically acceptable salt thereof.

7. A method of inhibiting adaptor associated kinase 1 (AAK1) activity, comprising contacting AAK1 with a compound of formula (I) 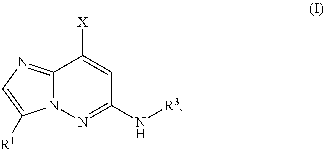
or a pharmaceutically acceptable salt thereof, wherein:
R¹ is selected from —C(O)NHR² and thienyl;
R² is selected from 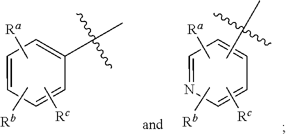
wherein R^{a} and R^{b} are independently selected from hydrogen, C₂-C₄ alkenyl, C₁-C₃alkoxy, C₁-C₃alkoxyC₁-C₃alkyl, C₁-C₃ alkyl, cyano, halo, C₁-C₃ haloalkyl, hydroxy, and C₁-C₃hydroxyalkyl; or, alternatively,
when R^{a} and R^{b} are on adjacent carbons, they, together with the carbon atoms to which they are attached, can optionally form a five-membered aromatic ring containing one or two nitrogen atoms;
R^{c} is a five-membered aromatic ring containing one, two, three, or four heteroatoms independently selected from nitrogen, oxygen, and sulfur, wherein the five-membered aromatic ring is optionally substituted with one group selected from C₁-C₄alkoxy, C₁-C₄ alkoxyC₁-C₄ alkyl, C₁-C₄alkyl, C₁-C₄ aminoalkyl, cyano, C₃-C₆ cycloalkyl, C₁-C₄ haloalkyl, C₁-C₄ hydroxyalkyl, nitro, and phenyl;
R³ is selected from 4-acylaminocyclohexyl, C₁-C₄-aminoalkyl, 2-aminocyclobutyl, 4-aminocyclohexyl, 3-aminocyclopentyl, 3-aminomethylcyclohexyl, 3-aminomethylcyclopentyl, 2-cyanocyclobutyl, 4-cyanocyclohexyl, cyanomethyl, 2-methylaminocyclobutyl, 4-methylaminocyclohexyl, 3-methylaminocyclopentyl, octahydrocyclopenta[c]pyrrolyl, 4-piperidyl, and 3-azabicyclo[3.2.1]octyl; and
X is selected from hydrogen, C₁-C₃alkylamino, C₃-C₆cycloalkylamino, and phenylamino, wherein the phenylamino is optionally substituted with one group selected from C₁-C₃alkoxy, C₁-C₃alkyl, cyano, and a five-membered aromatic ring containing one, two, or three heteroatoms independently selected from nitrogen, oxygen, and sulfur wherein the five-membered aromatic ring is optionally substituted with one C₁-C₃alkyl group.

8. The method of claim 7 wherein
R² is 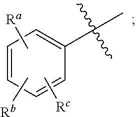
wherein R^{a} and R^{b} are hydrogen;
R^{c} is a five-membered aromatic ring containing one, two, three, or four 140 heteroatoms independently selected from nitrogen, oxygen, and sulfur, wherein the five-membered aromatic ring is optionally substituted with one group selected from C₁-C₄alkoxy, cyano, nitro, and phenyl; and
R³ is 4-aminocyclohexyl.
